(19) United States
(12) Patent Application Publication
(10) Pub. No.: US 2023/0172929 A1
(43) Pub. Date: Jun. 8, 2023

(54) NOVEL PERIPHERAL CANNABINOID-1 RECEPTOR ANTAGONISTS

(71) Applicant: Yissum Research Development Company of the Hebrew University of Jerusalem Ltd., Jerusalem (IL)

(72) Inventors: Joseph TAM, Jerusalem (IL); Amiram GOLDBLUM, Tel Aviv (IL); Shayma EL-ATAWNEH, Hura (IL); Shira HIRSH, Jerusalem (IL)

(73) Assignee: Yissum Research Development Company of the Hebrew University of Jerusalem Ltd., Jerusalem (IL)

(21) Appl. No.: 17/633,551

(22) PCT Filed: Aug. 6, 2020
(86) PCT No.: PCT/IL2020/050861

§ 371 Date: Feb. 7, 2022

Related U.S. Application Data
(60) Provisional application No. 62/884,551, filed on Aug. 8, 2019.

(51) Int. Cl. A61K 31/505; A61K 31/4045; A61K 31/167; A61K 31/426; A61K 31/381; A61K 31/18; A61K 31/404

(52) U.S. Cl. CPC A61K 31/505; A61K 31/4045; A61K 31/167; A61K 31/426; A61K 31/381; A61K 31/18; A61K 31/404

(57) ABSTRACT

The technology disclosed herein concerns compounds capable of binding to CB1Rs in the periphery and not in the CNS.

### TECHNOLOGICAL FIELD

This invention generally relates to cannabinoid-1 receptor antagonists, inverse agonists and allosteric modulators and their uses in medicine.

### BACKGROUND

The cannabinoid receptors (CBRs), belong to the superfamily of G-protein coupled receptors (GPCRs), include two members: the CB1 and CB2 receptors (CB1R and CB2R, respectively).

CB1R is mainly expressed in the central nervous system (CNS), but also in many peripheral tissues like the gastrointestinal (GI) tract, liver and others. CB2R is mainly associated with immune cells and may be found at a lower concentration in the CNS. Both receptors regulate a variety of central and peripheral physiological functions. They are therefore potential therapeutic targets for many disorders.

CB1R is involved in the overall homeostatic balance and regulation of food intake, fat accumulation, lipid, and glucose metabolism in the CNS as well as in periphery. Stimulation of the hypothalamic CB1Rs affects neuropeptides that regulate energy homeostasis, food intake and lipogenesis in visceral tissues. However, stimulation of the CB1R in the nucleus excites the dopaminergic reward pathway and thus increases the motivation to eat, as well as to smoke or intake drugs of abuse.

In fact, all the above-mentioned observations provided the motivation for testing such compounds as a potential treatment for obesity. However, the pharmaceutical industry hesitates to develop agents that target these receptors due to the CNS-mediated psychoactive effects induced by cannabinoids (e.g., Rimonabant).

Peripheral CB1R antagonists could also be used for the prevention and management of conditions related to obesity like cardiovascular disease, insulin resistance, dyslipidemia, fatty liver, hypertension, chronic inflammation, hypercoagulable/prothrombotic state and chronic kidney disease. All these disorders constitute the metabolic syndrome. Peripheral CB1R antagonists were found to prevent the development of obesity and its metabolic comorbidities such as insulin resistance, hepatic steatosis, nephropathy, and a combined treatment of peripheral CB1R antagonist and CB2R agonist was shown to abolish diabetes-induced albuminuria, inflammation, tubular injury, and renal fibrosis.

To conclude, there is a need for peripheral CB1R antagonists with good effectiveness against metabolic disorders but without the centrally mediated side effects, which are associated with this type of compounds.

### General Description

The inventors of the technology disclosed herein have developed a methodology whereby compounds which bind to CB1Rs in the periphery and not in the CNS, retain the therapeutic benefits of globally acting CB1R blockers without causing CNS-mediated side effects; thus, reviving the earlier prospect of CB1R blockade for the treatment of metabolic syndromes. To that end, the inventors have constructed models for CB1R modulation, e.g., antagonism, using machine learning “Iterative Stochastic Elimination” (ISE) algorithm (which among others serves for the discovery of highly active molecules), thus discovering a large group of CB1R antagonist candidates. Applying a set of rules to that group reduced it to a group of novel compounds that do not penetrate the blood-brain-barrier (BBB) and thus block the CB1R only in peripheral tissues, without causing centrally mediated side effects.

This novel group of compounds exhibited efficacy in affecting several features of the well-known metabolic syndrome.

Thus, in a first aspect of the presently disclosed invention, there is provided a compound for use in medicine, the compound herein being designated compound (1) through compound (14) is a compound of a structure selected from:

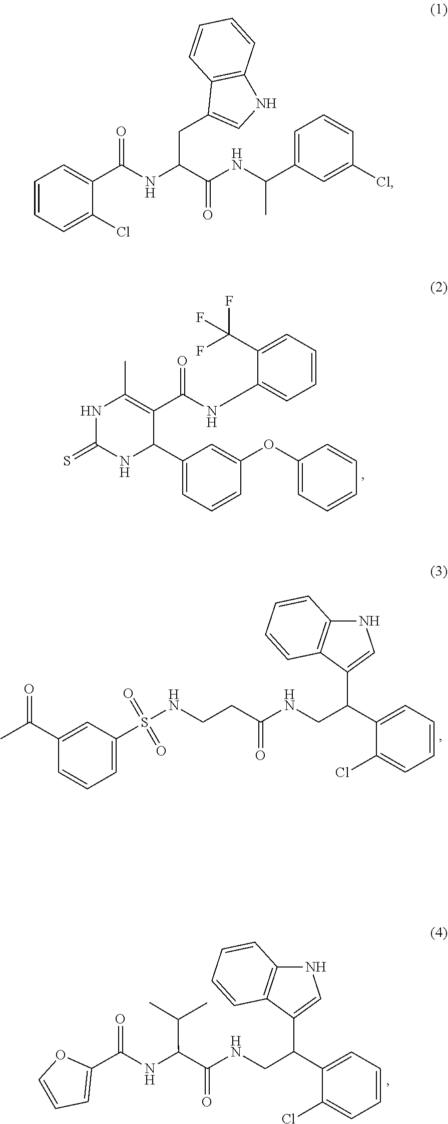 [SMILES: CC(NC(=O)C(Cc1c[nH]c2ccccc12)NC(=O)c1ccccc1Cl)c1cccc(Cl)c1 ; CC1=C(C(=O)Nc2ccccc2C(F)(F)F)C(c2cccc(Oc3ccccc3)c2)NC(=S)N1 ; CC(=O)c1cccc(S(=O)(=O)NCCC(=O)NCC(c2ccccc2Cl)c2c[nH]c3ccccc23)c1 ; CC(C)C(NC(=O)c1ccco1)C(=O)NCC(c1ccccc1Cl)c1c[nH]c2ccccc12]

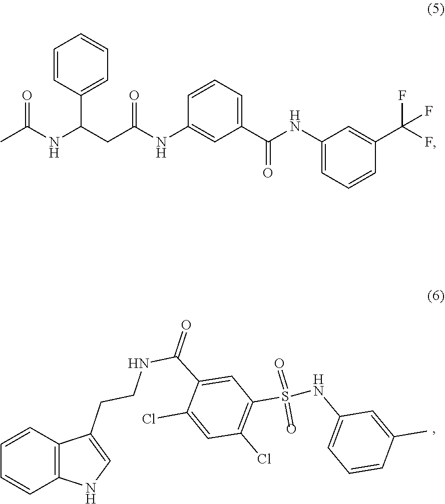

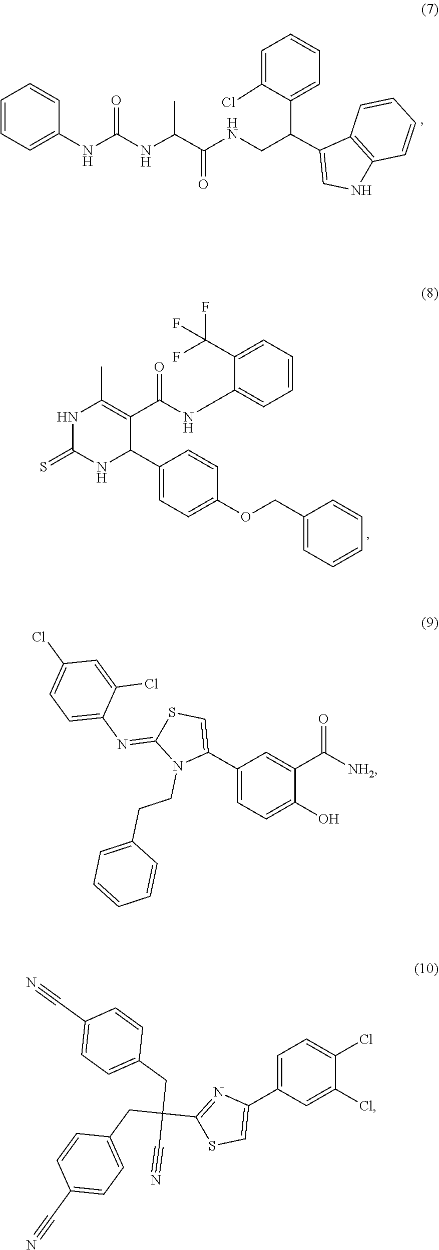 [SMILES: CC(NC(=O)Nc1ccccc1)C(=O)NCC(c1ccccc1Cl)c1c[nH]c2ccccc12 ; CC1=C(C(=O)Nc2ccccc2C(F)(F)F)C(c2ccc(OCc3ccccc3)cc2)NC(=S)N1 ; NC(=O)c1cc(-c2cs/c(=N\c3ccc(Cl)cc3Cl)n2CCc2ccccc2)ccc1O ; N#Cc1ccc(CC(C#N)(Cc2ccc(C#N)cc2)c2nc(-c3ccc(Cl)c(Cl)c3)cs2)cc1]

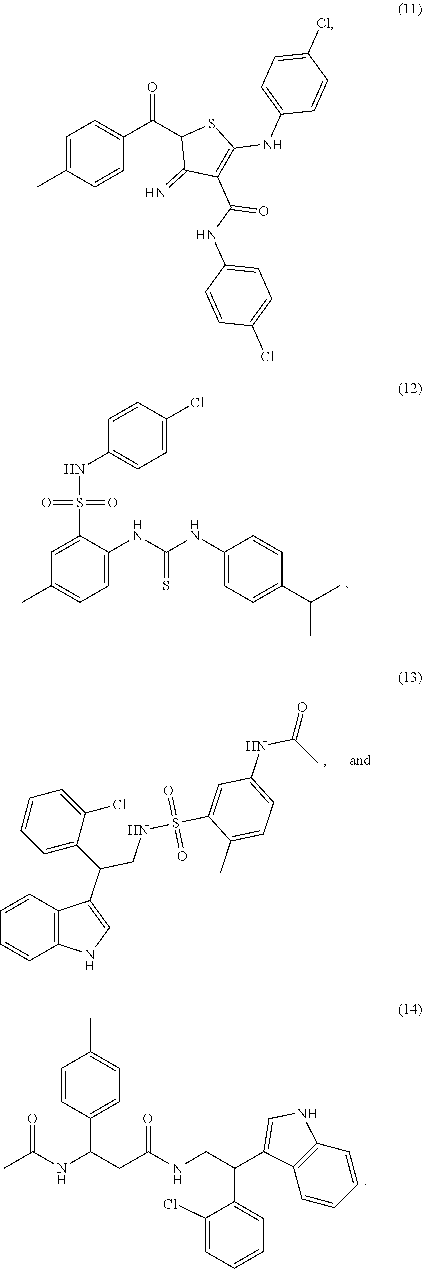

When referring to any one of compounds (1) through (14), each compound should be regraded as an independent selection. Thus, when referring to the compounds, reference is to compound herein designated compound (1) or (2) or (3) or (4) or (5) or (6) or (7) or (8) or (9) or (10) or (11) or (12) or (13) or (14).

The invention further provides a compound for use in medicine, the compound being a compound selected from compound (4) and compound (8). In some embodiments, the compound is:

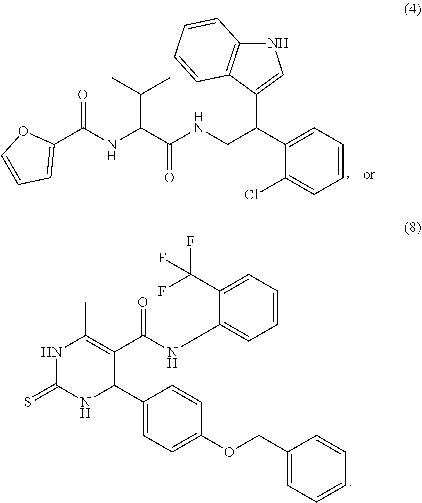 [SMILES: CC(C)C(NC(=O)c1ccco1)C(=O)NCC(c1ccccc1Cl)c1c[nH]c2ccccc12 ; CC1=C(C(=O)Nc2ccccc2C(F)(F)F)C(c2ccc(OCc3ccccc3)cc2)NC(=S)N1]

In some embodiments, amongst the compounds herein designated compound (1) through compound (14), the compound is a compound herein designated compound (4) or compound (8).

Also provided is a compound of the general formula (I) for use in medicine:

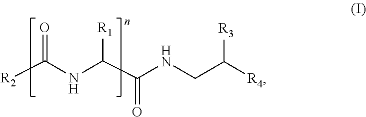

wherein in a compound of formula (I):

n is an integer between 1 and 3;

R₁ is a C₁-C₅alkyl;

and

each of R₂, R₃ and R₄, independently of the other, is a C₆-C₁₀aryl, a C₅-C₁₀heteroaryl or a C₅-C₁₀carbocycle.

In some embodiments, n is 1, 2 or 3. In some embodiments, n is 1.

In some embodiments, R₁ is a methyl or an ethyl or a propyl or a butyl or a pentyl. In some embodiments, R₁ is a methyl or an ethyl or a propyl.

In some embodiments, R₁ is 2-propyl.

In some embodiments, each of R₂, R₃ and R₄, independently of the other, is selected from a phenyl, a substituted phenyl, furanyl, a substituted furanyl, pyrronyl, a substituted pyrrolyl, thiophenyl and substituted thiophenyl.

In some embodiments, each of R₂, R₃ and R₄, independently of the other, is selected from a fused aryl and a fused heteroaryl.

In some embodiments, each of R₂, R₃ and R₄, independently of the other, is an optionally substituted indolyl, an optionally substituted benzofuranyl, an optionally substituted benzothiophenyl.

Also provided is a compound of formula (II) for use in medicine:

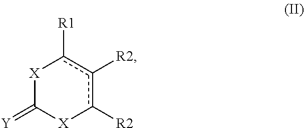

wherein

each X is a heteroatom selected from O, NH and S;

Y is a heteroatom selected from O, NH and S or is a tertiary N atom;

R₁ is a C₁-C₅alkyl;

R₂ is a —(C═O)NH—R₃;

R₃ is a C₆-C₁₀aryl or a C₅-C₁₀heteroaryl; and

one of the bonds designated [symbol] is a double bond and the other is a single bond.

In some embodiments, each X is the same.

In some embodiments, each X is NH or O.

In some embodiments, each X is NH.

In some embodiments, one or both of X are a tertiary N group.

In some embodiments, Y is S.

In some embodiments, R₁ is a methyl, ethyl, propyl, butyl or pentyl. In some embodiments, R₁ is methyl, ethyl or propyl.

In some embodiments, R₂ is a —(C═O)NH-phenyl, wherein the phenyl is optionally substituted.

In some embodiments, the phenyl is substituted by at least one halogen or a group comprising one or more halogen atoms. In some embodiments, the group comprising one or more halogen atoms is substituted by 1, 2, 3 or more F atoms. In some embodiments, the group comprising one or more halogen atoms is a trifluorinated methyl.

In some embodiments, R₃ is a substituted phenyl. In some embodiments, R₃ is a phenyl substituted with a group selected from a hydroxide group, an ether group and an ester group.

In some embodiments, R₃ is a phenyl substituted ether group.

In some embodiments, R₃ is a phenyl substituted with a group having the structure —O—(CH₂)m-C₆-C₁₀aryl or —O—(CH₂)m-C₅-C₁₀heteroaryl, wherein m is an integer between 0 and 3 and C₆-C₁₀aryl is optionally a phenyl.

In some embodiments, the group —O—(CH₂)m-C₆-C₁₀aryl is —O—(CH₂)m-phenyl.

In some embodiments, in the group —O—(CH₂)m-C₆-C₁₀aryl, m is 1.

As used herein, the optional substitution referred to above, in groups constructing compound of formula (I) or formula (II), may be a substitution by an alkyl, an hydroxyl, an amine, a halide, an ether, an ester, a thiol, a sulfide, an aryl, or any other atom or group, as known in the art. The substitution may be of a single atom or a group of atoms or may be of multiple atoms or groups of atoms which may be the same or different.

It is to be understood that the compounds provided herein may contain chiral centers. Such chiral centers may be of either the (R) or (S) configuration or may be a mixture thereof. Thus, compounds provided herein may be enantiomerically pure, or in stereoisomeric or diastereomeric mixtures. In the case of amino acid residues, such residues may be of either the L- or D-form. One of skill in the art will recognize that administration of a compound in its (R) form is equivalent, for compounds that undergo epimerization in vivo, to administration of the compound in its (S) form.

As used herein, an alkyl contains the specified number of carbon atoms, e.g., between 1 and 5, inclusive, and are straight or branched. Exemplary alkyl groups include, but are not limited to, methyl, ethyl, propyl, isopropyl, isobutyl, n-butyl, sec-butyl, tert-butyl, and others.

As used herein, a “carbocyclyl” or a “cycloalkyl” refers to a saturated mono- or multi-cyclic ring system, in certain embodiments of 3 to 10 carbon atoms, in other embodiments of 5 to 10 carbon atoms. The ring system of the cycloalkyl may be composed of one ring or two or more rings which may be joined together in a fused, bridged or spiro-connected fashion.

As used herein, “aryl” refers to aromatic monocyclic or multicyclic groups containing from 6 to 10 carbon atoms. Aryl groups include, but are not limited to groups such as unsubstituted or substituted fluorenyl, unsubstituted or substituted phenyl, and unsubstituted or substituted naphthyl, or others as mentioned herein.

As used herein, “heteroaryl” refers to a monocyclic or multicyclic aromatic ring system, in certain embodiments, of about 5 to about 10 atoms where one or more, in one embodiment 1 to 3, of the atoms in the ring system is a heteroatom, that is, an element other than carbon, including but not limited to, nitrogen, oxygen or sulfur. The heteroaryl group may be optionally fused to a benzene ring. Heteroaryl groups include, but are not limited to, furyl, imidazolyl, pyrimidinyl, tetrazolyl, thienyl, pyridyl, pyrrolyl, thiazolyl, isothiazolyl, oxazolyl, isoxazolyl, triazolyl, quinolinyl and isoquinolinyl,

As used herein, “halo”, “halogen” or “halide” refers to F, Cl, Br or I.

As used herein, any substituent or variant discussed or mentioned herein, is in accord with their common usage, recognized abbreviations, or the IUPAC-IUB Commission on Biochemical Nomenclature (see, (1972) Biochem. 11:942-944).

Compounds used in accordance with the invention may be presented as pharmaceutically acceptable acid addition salts. Such salts may include salts derived from inorganic acids such as hydrochloric, nitric, phosphoric, sulfuric, hydrobromic, hydriodic, phosphorous, and the like, as well as the salts derived from organic acids, such as aliphatic mono- and dicarboxylic acids, phenyl-substituted alkanoic acids, hydroxy alkanoic acids, alkanedioic acids, aromatic acids, aliphatic and aromatic sulfonic acids, etc. These salts thus include sulfate, pyrosulfate, bisulfate, sulfite, bisulfate, nitrate, phosphate, monohydrogenphosphate, dihydrogenphosphate, metaphosphate, pyrophosphate, chloride, bromide, iodide, acetate, propionate, caprylate, isobutyrate, oxalate, malonate, succinate, suberate, sebacate, fumarate, maleate, mandelate, benzoate, chlorobenzoate, methylbenzoate, dinitrobenzoate, phthalate, benzenesulfonate, toluenesulfonate, phenylacetate, citrate, lactate, maleate, tartrate, methanesulfonate, and the like.

Also contemplated are salts of amino acids such as arginate and the like and gluconate, galacruronate (see, for example, Berge S. M., et al., “Pharmaceutical Salts,” J. of Pharmaceutical Science, 66:1-19 (1977)). The acid addition salts of basic compounds are prepared by contacting the free base form with a sufficient amount of the desired acid to produce the salt in the conventional manner. The free base form may be regenerated by contacting the salt form with a base and isolating the free base in the conventional manner. The free base forms differ from their respective salt forms somewhat in certain physical properties such as solubility in polar solvents, but otherwise the salts are equivalent to their respective free base for purposes of the present invention. Pharmaceutically acceptable base addition salts are formed with metals or amines, such as alkali and alkaline earth metals or organic amines. Examples of metals used as cations are sodium, potassium, magnesium, calcium, and the like. Examples of suitable amines are N,N′-dibenzylethylenediamine, chloroprocaine, choline, diethanolamine, ethylenediamine, N-methylglucamine, and procaine (see, for example, Berge S. M., et al., “Pharmaceutical Salts,” J. of Pharmaceutical Science, 66:1-19 (1977)).

The base addition salts of acidic compounds may be prepared by contacting the free acid form with a sufficient amount of the desired base to produce the salt in the conventional manner. The free acid form may be regenerated by contacting the salt form with an acid and isolating the free acid in the conventional manner. The free acid forms differ from their respective salt forms somewhat in certain physical properties such as solubility in polar solvents, but otherwise the salts are equivalent to their respective free acid for purposes of the present invention.

Peripheral CB1R antagonists could be used for the prevention and management of conditions related to obesity, e.g., cardiovascular diseases, insulin resistance, dyslipidemia, hypertension, fatty liver, chronic inflammation, hypercoagulable state and chronic kidney disease. This group of disorders constitutes the so-called metabolic syndrome. Therefore, in some embodiments, each of the designated compound (1) through compound (14), independently, or any compound of the general formula (I) or (II) may be used in preventing or treating a metabolic syndrome or its related disorders as mentioned hereinbelow.

The metabolic disorders may include obesity, insulin resistance, diabetes, coronary heart disease, liver steatosis and cirrhosis, dyslipidaemia, hypertension, chronic inflammation, a hypercoagulable state, acute kidney disease and chronic kidney disease.

In some embodiments, each one of the designated compounds (1) through (14), independently, or a compound of the formula (I) or (II), or compound (4) or compound (8), alone or in combination, may be used for treating a subject for the purpose of reducing the subject's body fat or body weight, or treating insulin resistance, or treating diabetes, or reducing or controlling high blood pressure, or improving a poor lipid profile with elevated LDL cholesterol, low HDL cholesterol, and elevated triglycerides, or treating acute and chronic kidney injury, or treating a metabolic syndrome.

In another one of its aspects, each one of the compounds designated compounds (1) through (14), independently, or a compound of the formula (I) or (II) may be used for the preparation of a pharmaceutical composition.

As used herein, the “pharmaceutical composition” comprises a therapeutically effective amount of a compound disclosed herein, optionally together with suitable additives such as diluents, preservatives, solubilizers, emulsifiers, adjuvant and/or carriers. The compositions may be liquids or lyophilized or otherwise dried formulations and include diluents of various buffer content (e.g.; Tris-HCL, acetate, phosphate), pH and ionic strength, additives such as albumin or gelatin to prevent absorption to surfaces, detergents (e.g., Tween 20, Tween 80, Pluronic F68, bile acid salts), solubilizing agents (e.g., glycerol, polyethylene glycerol), anti-oxidants (e.g., ascorbic acid, sodium metabisulfite), preservatives (e.g., Thimerosal, benzyl alcohol, parabens), and others.

The amount of the one or more compounds that is contained in a composition of the invention is effective to achieve the desired therapeutic effect as described herein, depending, inter alia, on the type and severity of the disease to be treated or prevented and the regime used. The effective amount is typically determined in appropriately designed clinical trials (dose range studies) and the person versed in the art will know how to properly conduct such trials in order to determine the effective amount. As generally known, an effective amount depends on a variety of factors including the affinity of the ligand to the receptor, its distribution profile within the body, a variety of pharmacological parameters such as half life in the body, on undesired side effects, if any, on factors such as age and gender, and others.

Compositions of the invention may be administered by any mode known in the art. Accordingly, pharmaceutical compositions of the present invention may be adapted for oral, aerosol, parenteral, subcutaneous, intravenous, intramuscular, interperitoneal, rectal and vaginal administration. In some embodiments, compounds and compositions of the invention are adapted for oral administration.

Compositions suitable for oral administration can comprise of (a) liquid solutions, such as an effective amount of the compound dissolved in diluents, such as water, saline, or orange juice; (b) capsules, sachets, tablets, lozenges, and troches, each containing a predetermined amount of the active ingredient, as solids or granules; (c) powders; (d) suspensions in an appropriate liquid; and (e) suitable emulsions or self-emulsifying formulations. Liquid formulations may include diluents, such as water and alcohols, for example, ethanol, benzyl alcohol, and the polyethylene alcohols, either with or without the addition of a pharmaceutically acceptable surfactant, suspending agent, or emulsifying agent. Capsule forms can be of the ordinary hard- or soft-shelled gelatin type containing, for example, surfactants, lubricants, and inert fillers. Tablet forms can include one or more of lactose, sucrose, mannitol, corn starch, potato starch, alginic acid, microcrystalline cellulose, acacia, gelatin, guar gum, colloidal silicon dioxide, croscarmellose sodium talc, magnesium stearate, calcium stearate, zinc stearate, stearic acid, and other excipients, colorants, diluents, buffering agents, disintegrating agents, moistening agents, preservatives, flavoring agents, and pharmacologically compatible carriers. Lozenge forms can comprise the active ingredient in a flavor, usually sucrose and acacia or tragacanthin, as well as pastilles comprising the active ingredient in an inert base, such as gelatin and glycerin, or sucrose and acacia, emulsions, gels, and the like containing, in addition to the active ingredient, such carriers as are known in the art.

Compositions suitable for parenteral administration include sterile nanoemulsions, aqueous and non-aqueous, isotonic sterile injection solutions, which can contain anti-oxidants, buffers, bacteriostats, and solutes that render the formulation isotonic with the blood of the intended recipient, and aqueous and non-aqueous sterile suspensions that include suspending agents, solubilizers, thickening agents, stabilizers, and preservatives.

Compounds of the invention can be administered in a physiologically acceptable diluent in a pharmaceutical carrier, such as a sterile liquid or mixture of liquids, including water, saline, aqueous dextrose and related sugar solutions, an alcohol, such as ethanol, isopropanol, or hexadecyl alcohol, glycols, such as propylene glycol or polyethylene glycol, glycerol ketals, such as 2,2-dimethyl-1,3-dioxolane-4-methanol, ethers, such as poly(ethyleneglycol) 400, an oil, a fatty acid, a fatty acid ester or glyceride, or an acetylated fatty acid glyceride with or without the addition of a pharmaceutically acceptable surfactant, such as a soap or a detergent, suspending agent, such as pectin, carbomers, methylcellulose, hydroxypropylmethylcellulose, or carboxymethylcellulose, or emulsifying agents and other pharmaceutical adjuvants. Oils, which can be used in parenteral formulations include petroleum, animal, vegetable, or synthetic oils. Specific examples of oils include peanut, soybean, sesame, cottonseed, corn, olive, petrolatum, and mineral. Suitable fatty acids for use in parenteral formulations include oleic acid, stearic acid, and isostearic acid.

Compounds of the present invention may be made into injectable formulations. The requirements for effective pharmaceutical carriers for injectable compositions are well known to those of ordinary skill in the art. See Pharmaceutics and Pharmacy Practice, J. B. Lippincott Co., Philadelphia, Pa., Banker and Chalmers, eds., pages 238-250 (1982), and ASHP Handbook on Injectable Drugs, Toissel, 4th ed., pages 622-630 (1986).

In another one of its aspects the invention contemplates a pharmaceutical composition comprising at least one of any of the designated compounds (1) through (14) or a compound of the general formula (I) or (II), or compound (4) or compound (8) or a combination of two or more of these compounds.

In some embodiments, pharmaceutical compositions used herein may be used for preventing or treating a metabolic syndrome or its related disorders. In some embodiments, the metabolic syndrome or its related disorder is selected from the disorders described hereinabove. In some embodiments, the pharmaceutical compositions may be used for treating a subject to reduce body fat, or to reduce body weight, or to treat insulin resistance, or to treat diabetes, or to reduce or control high blood pressure, or to improve a poor lipid profile with elevated LDL cholesterol, low HDL cholesterol, and elevated triglycerides, or to treat acute and chronic kidney injury, or to treat a metabolic syndrome.

Also contemplated is a use of one of the hereinabove designated compounds in a method of treatment of a subject. In some embodiments, the method is for preventing or treating a metabolic syndrome or disorder, as defined.

In yet another one of its aspects, the invention contemplates a method of treating a disease or disorder in a subject, the method comprising administering to the subject a compound designated compound (1) through (14) or a compound of the general formula (I) or (II) or compound (4) or compound (8) or combinations thereof. In some embodiments, the disease or disorder is a metabolic syndrome or disorder. In some embodiments, the metabolic syndrome or disorder may be selected from obesity, insulin resistance, diabetes, coronary heart disease, liver steatosis and cirrhosis, dyslipidaemia, hypertension, chronic inflammation, a hypercoagulable state, acute kidney disease and chronic kidney disease. In some embodiments, the method is for reducing body fat, reducing body weight, treating insulin resistance, treating diabetes, reducing or controlling high blood pressure, or improving a poor lipid profile with elevated LDL cholesterol, low HDL cholesterol, and elevated triglycerides, or treating acute and chronic kidney injury, or the metabolic syndrome.

As indicated herein, the inventors have found that each one of the compounds designated herein compound (1) through compound (14) can bind to each of the peripherally restricted CB1 or CB2 receptors to induce inhibition, modulation or activation of the receptor(s). Furthermore, the inventors found that each of said compounds can act on CB1 and CB2 receptors in different ways. Therefore, the invention further provides a modulator of a peripherally restricted CB1 and/or CB2 receptor, wherein the modulator is a compound designated compound (1) through compound (14).

A “CB1 or CB2 receptor modulator” is a compound according to the invention which in most general terms can modify a biological function of a peripheral CB1 or CB2 receptor. Receptor modulator can be any one of the following: receptor agonist, receptor antagonist, receptor partial agonist, inverse agonist or an allosteric modulator. Such ligand/compound can alter the biological function of the receptor, therefore prevention or treatment of a variety of metabolic syndromes can be achieved. The “peripherally restricted CB1 or CB2 receptors” are receptors present in peripheral organs and tissues, including the adipose tissues, the liver, skeletal muscles, pancreatic (3-cells and the kidneys, excluding the receptors which appear in the CNS.

The invention thus further provides use of any one of the compounds designated compound (1) through compound (14) for modulating activity a peripherally restricted CB1 and/or CB2 receptor.

In another aspect the invention provides an inhibitor of a peripherally restricted CB1 and/or CB2 receptor, wherein the inhibitor is a compound designated compound (1) through compound (14).

A “CB1 or CB2 receptor blocker or antagonist or neutral antagonist or inhibitor” is a compound according to the invention which in most general terms partially or fully blocks, inhibits, or neutralizes a biological function of a peripheral CB1 or CB2 receptor. By partially or fully blocking, inhibiting, or neutralizing a biological function of the receptor, prevention or treatment of a variety of metabolic syndromes can be achieved.

In another one of its aspects the invention further provides use of any one of the compounds designated compound (1) through compound (14) for inhibiting activity of a peripherally restricted CB1 and/or CB2 receptor. In some embodiments, the compound is compound (4) or compound (8).

In yet another one of its aspects, the invention provides a neutral antagonist of a peripherally restricted CB1 and/or CB2 receptor, wherein the neutral antagonist is a compound designated compound (1) through compound (14). In some embodiments, the compound is compound (4) or compound (8).

In another one of its aspects the invention further provides use of any one of the compounds designated compound (1) through compound (14) as a neutral antagonist of any one of the peripherally restricted CB1 and/or CB2 receptors. In some embodiments, the compound is compound (4) or compound (8).

In yet another one of its aspects, the invention provides a blocker of a peripherally restricted CB1 and/or CB2 receptor, wherein the blocker is a compound designated compound (1) through compound (14). In some embodiments, the compound is compound (4) or compound (8).

In another one of its aspects the invention further provides use of any one of the compounds designated compound (1) through compound (14) for blocking activity of any one of the peripherally restricted CB1 and/or CB2 receptors. In some embodiments, the compound is compound (4) or compound (8).

In yet another one of its aspects, the invention provides an inverse agonist of a peripherally restricted CB1 and/or CB2 receptor, wherein the inverse agonist is a compound designated compound (1) through compound (14). In some embodiments, the compound is compound (4) or compound (8).

As used herein, the term “CB1 or CB2 receptor inverse agonist” is a compound according to the invention which in most general terms induces a pharmacological response which is opposite to that of an agonist. Thus, decreases the activity of CB1 or CB2 receptors below the basal level activity. By the activity of said receptors below the basal level, prevention or treatment of a variety of metabolic syndromes can be achieved.

In another one of its aspects the invention further provides use of any one of the compounds designated compound (1) through compound (14) as an inverse agonist of any one of the peripherally restricted CB1 and/or CB2 receptors. In some embodiments, the compound is compound (4) or compound (8).

In some embodiments, any one of the compounds can act on any one of the peripherally restricted CB1 and/or CB2 receptors as a neutral antagonist, as an inverse agonist or as a modulator. In other embodiments, said compounds can act as neutral antagonists or inverse agonists. In some embodiments, the compound is compound (4) or compound (8).

In yet another one of its aspects, any one of the peripherally restricted CB1 and/or CB2 receptors modulator or blocker or inhibitor or antagonist or neutral antagonist or inverse agonist is for treating or preventing a metabolic syndrome or disorder. In some embodiments, the compound is compound (4) or compound (8).

As used herein in reference to any aspects and embodiments of the invention, the term “treatment” and the term “prevention” refer to the administering of a therapeutically effective amount of a compound or a composition of the invention which is effective to ameliorate undesired symptoms associated with a disease, to prevent the manifestation of such symptoms before they occur, to slow down the progression of the disease, slow down the deterioration of symptoms, to enhance the onset of remission period, slow down the irreversible damage caused in the progressive chronic stage of the disease, to delay the onset of said progressive stage, to lessen the severity or cure the disease, to improve survival rate or more rapid recovery, or to prevent the disease form occurring or a combination of two or more of the above.

Thus, an effective amount of a compound used in accordance with the invention aims at the treatment and/or prevention of a metabolic syndrome, more specifically at reducing a subject's body fat or body weight, or treating insulin resistance, or treating diabetes, or reducing or controlling high blood pressure, or improving a poor lipid profile with elevated LDL cholesterol, low HDL cholesterol, and elevated triglycerides, or treating acute and chronic kidney injury, or treating a metabolic syndrome, which may be characterized by abdominal obesity, elevated blood pressure, elevated fasting plasma glucose, high serum triglycerides, and/or low high-density cholesterol levels.

The invention further provides a kit comprising a compound as disclosed herein and instructions of use. In some embodiments, the compound is compound (4) or compound (8).

In some embodiments, the kit is a medical package comprising instructions of using the compound for any of the medicinal purposes disclosed herein.

### BRIEF DESCRIPTION OF THE DRAWINGS

In order to better understand the subject matter that is disclosed herein and to exemplify how it may be carried out in practice, embodiments will now be described, by way of non-limiting example only, with reference to the accompanying drawings, in which:

FIG. 1 depicts the Tanimoto coefficient histograms for models A-D. Comparing the hits and the active molecules used to build each model.

FIG. 2 shows the ADP for the models. The randoms are selected from the same regions of chemical space of the known actives (i.e., “Applicability domain” (APD) calculated by the average ±2 standard deviations of 4 properties of the actives: molecular weight, computed lipophilic character, number of hydrogen bond donors and acceptors).

FIG. 3 shows the descriptors type distribution of the different models A-D.

FIG. 4 presents the scatter plots of the internal test sets distribution in each model A-D.

FIG. 5 presents Tanimoto distribution for models A-D.

FIG. 6 shows the distribution of peripheral properties for the ˜500 hits from VS.

FIG. 7 presents output of screening the 15 hits through GPCRs activity models.

FIGS. 8A-B present brain and serum levels following an administration of Compound 8 mix at different doses (3, 10 and 30 mg/kg). FIG. 8A shows drug levels in the brain. FIG. 8B shows drug levels in serum.

FIGS. 9A-E present brain, serum, liver, kidney, and fat levels following an administration of Compound 8 (enantiomer R) at different doses (3, 10, 30 mg/kg). FIG. 9A shows drug levels in the brain. FIG. 9B shows drug levels in serum. FIG. 9C shows drug levels in the liver. FIG. 9D shows drug levels in kidney. FIG. 9E shows drug levels in fat tissue.

FIGS. 10A-E present brain, serum, liver, kidney, and fat levels following an administration of the Compounds 4-RR and 4-SR at different doses (10, 20, 50 mg/kg).

FIG. 10A presents drug levels in the brain. FIG. 10B presents drug levels in serum. FIG. 10C presents drug levels in liver. FIG. 10D presents drug levels in kidney. FIG. 10E presents drug levels in fat.

FIGS. 11A-D present the effects of Compound 8-R and Rimonabant in inducing CB1 receptor-mediated hyperactivity.

FIGS. 12A-D present the effects of Compounds 4-RR or 4-SR and Rimonabant in inducing CB1 receptor-mediated hyperactivity.

### DETAILED DESCRIPTION OF EMBODIMENTS

Data and Methods

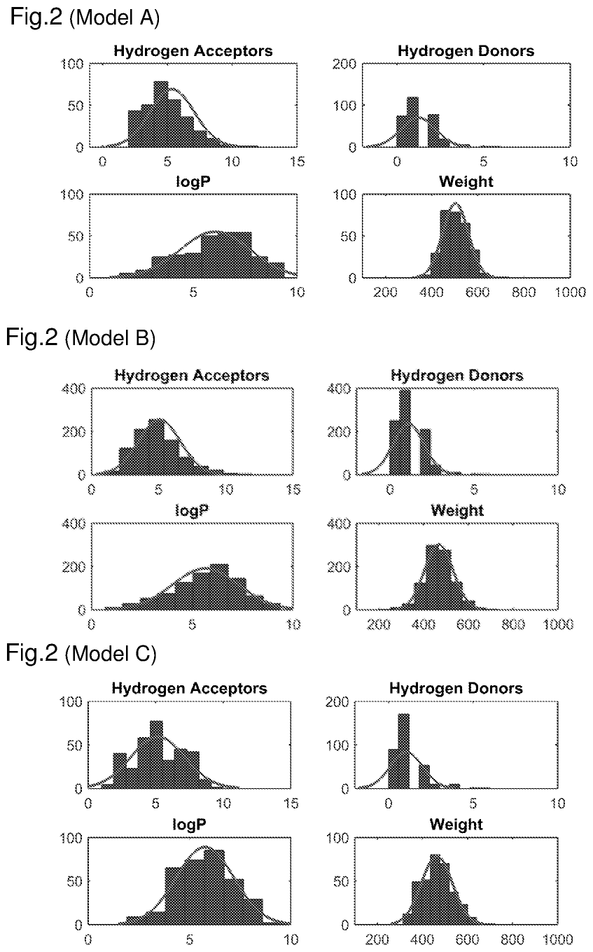

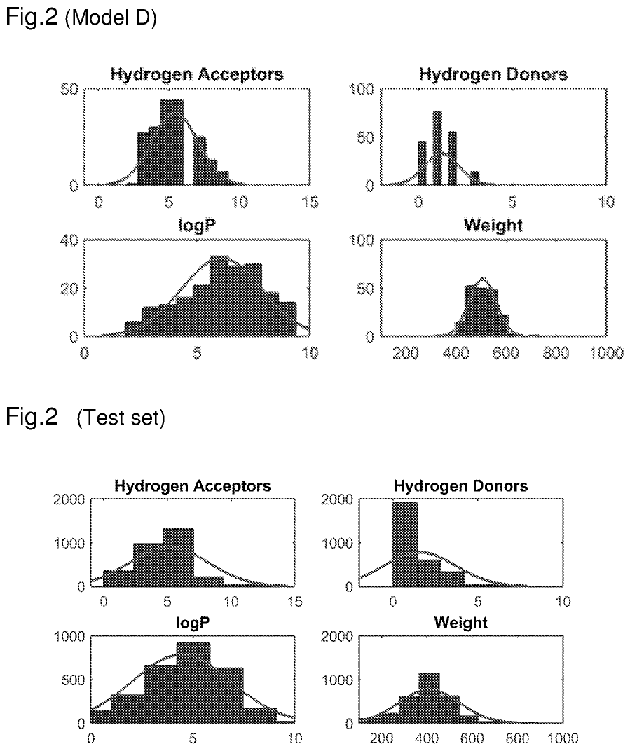

Data preparation: Known active molecules with antagonistic activity were taken from Chembl database (http://www.ebi.ac.uk/chembldb/) to form the core of our “learning set”. We included among the “actives” molecules for which either IC₅₀ or Kᵢ values were reported. Activity duplicates were removed (keeping the molecules with the lesser reported activity), as well as other possible sources of error (such as “Outside typical range” or “potential transcription error”). Low activities (greater than 100 μM) were excluded, and only molecules that have a “confidence score” above 7 were kept (this score is given by Chembl and reflects both the type of target assigned to a particular assay and the confidence that the target assigned is the correct target for that assay). In order to mimic in silico the standards of High-throughput screening (HTS) where the rate of discovery is about 1:1000 hits: screening set we “dilute” the learning set of actives with a huge set (100-fold) of randomly picked molecules. We choose the randoms from the same regions of chemical space of the known actives (i.e., “Applicability domain” (APD) calculated by the average ±2 standard deviations of 4 properties of the actives: molecular weight, computed lipophilic character, number of hydrogen bond donors and acceptors). The random molecules were selected from the ZINC database (containing overall 17,901,107 molecules) or the Enamine database (from a total of 2,170,859 molecules), see Table 1 and FIG. 2 for APD of the models.

TABLE 1
Physical properties of the learning sets.

| | Molecular | | Hydrogen | Hydrogen |
|---|---|---|---|---|
| Dataset | weight | LogP | acceptors | donors |
| IC₅₀ data | 199-732 | 0.6-9.4 | 1-12 | 0-6 |
| Kᵢ data | 258-1097 | 0.5-17 | 1-11 | 0-6 |

The final numbers of “actives”, following filtration as mentioned above, of Chembl picked antagonists with measured IC₅₀ was 906 molecules (IC₅₀=0.2-89,000 nM) and 1903 molecules with measured Kᵢ (Kᵢ=0.09-34,673 nM). Two of the models were built with the “inactives” (“decoys”), picked randomly from the ZINC database (Models A and C) and one model was built by picking randoms from the Enamine database (Model B). The inactivity is an assumption and those three models were based on a dilution of actives with a large number of inactives. Model D was a “High vs. Low” model, taking into account only active molecules among the 906 with IC₅₀ values: highly actives were those with IC₅₀ lower than 5 nM, and low actives were those with IC₅₀ greater than 500 nM. In this model, dilution is not required. Model A consisted of highly active molecules with IC₅₀ below 10 nM, Model C with highly active molecules with Kᵢ below 10 nM, while Model B was built from all active molecules. An external test set of CB1R antagonists was generated from Chembl (January, 2019) by excluding molecules used in the learning set, and contains 2970 molecules, out of which 2098 are actives and 872 are inactives (Table 2).

TABLE 2
The number of molecules used to build the learning and external test sets.

| | | | | | External |
|---|---|---|---|---|---|
| Dataset | Model A | Model B | Model C | Model D | test set |
| Number of | 296 | 906 | 332 | High < 5nM = | 2098 actives |
| active molecules | | | | 192 | |
| Number of | 33000 | 90000 | 35000 | Low > 500 nM = | 872 inactives |
| decoy | (ZINC | (Enamine | (ZINC | 233 | |
| molecules* | database) | database) | database) | | |

The learning set, the external test set, and the commercial libraries for virtual screening (VS) were “washed” (from counter ions) and 2D descriptors (185 physicochemical properties) were calculated for each molecule by MOE software (v. 2011.10). Reactive and mutagenic molecules, based on the calculated descriptors were removed from the learning set. Similarity calculations (Tanimoto coefficient) were done using fingerprints generated by RDKit toolkit in KNIME platform (v 2.10).

Building activity models using ISE: Iterative Stochastic Elimination (ISE) is an already established algorithm used by us for predicting molecular activities and picking molecular candidates for experimental testing. It is a generic algorithm, which finds large sets of good solutions in extremely complex combinatorial problems. It has been recently applied mostly to molecular discovery by optimizing the differences in physicochemical properties between two classes—active molecules and inactive (or, less active) ones. We construct—by random choice out of the physicochemical properties—filters made out, each, of five properties and examine whether the learning set molecules fit the values of those properties in a specific filter. It is thus easy to identify if actives are true positives (TP, if they pass the filter) or false negatives (FN, if they do not pass). Similarly, whether inactives are true negatives (TN, failing to pass the filter) or false positives (FP, those inactives that pass the filter). We feed the percentage of each of those categories into the Matthews Correlation Coefficient equation (Eq. 1, −1<MCC<1). Repeating that process for very many filters, we determined which properties are consistently associated with worst MCC values and do not contribute to best MCC values and eliminated those values. After a few iterations, we reached a point from which exhaustive calculations of all filters is possible, as the number of remaining combinations has been reduced. The top few hundred filters remained as our model. Having a final model composed of filters (five ranges of descriptors) allowed us to screen millions of molecules and to score them, by adding to each molecule the TP/FP score if it passes a filter or subtracting that number if it fails to pass. Further details may be found in the references mentioned above.

$$Eq . 1 The Matthews Correlation Coefficient equation .$$

$$MCC = \frac{TP * TN - FP * FN}{\sqrt{( TP + FP ) ( TP + FN ) ( TN + FP ) ( TN + FN )}}$$

Criteria for peripheral action: To limit our discovered molecules to candidates for peripheral action, and to lower the probability to enter the CNS by passing the blood-brain barrier (BBB), we applied filtration criteria (Table 3). The first column lists the features that distinguish CNS drugs, as well as features of the selective CB1R antagonist Rimonabant and our criteria for peripheral candidates.

TABLE 3
Peripheral filtration criteria.

| CNS Drugs with low to | | |
|---|---|---|
| sub nanomolar activity | Rimonabant | Our criteria |
| 1. Lower hydrophobicity | 1. cLogP = 6.28 | 1. cLogP >4 |
| (clogP <5) | | |
| 2. Molecular weight | 2. MW = 464 | 2. MW >450 |
| (MW) <450 | | |
| 3. Polar surface area-PSA | 3. PSA(A²) = 50 | 3. PSA(A²) >70 |
| (A²) <70 | | |
| 4. Number of H-bond donors | 4. HBD = 1 | 4. HBD >3 |
| (HBD) <3 | | |
| 5. Number of H-bond | | |
| acceptors <7 | | |
| 6. Number of rotatable | | |
| bonds <8 | | |

Radioligand binding assays: Binding to CB1R and CB2R was assessed in competition displacement assays using [3^{H}]CP-55,940 as the radioligand and crude membranes from mouse brain for CB1R or human cell membrane for CB2R, as reported previously. All data were in triplicates with Ki values determined from three independent experiments.

[³⁵S]GTPγS binding: Mouse brains (CB1R) or human cell membranes (CB2R) were dissected and P2 membranes prepared and resuspended at ˜2 μg protein/μL in 1 mL assay buffer (50 mM Tris HCl, 9 mM MgCl2, 0.2 mM EGTA, 150 mM NaCl; pH 7.4). Ligand-stimulated [³⁵S]GTPγS binding was assayed as described previously. Briefly, membranes (10 μg protein) were incubated in assay buffer containing 100 μM GDP, 0.05 nM [³⁵S]GTPγS, test compounds (HU-210, CP55,940 and tested molecules) at 10 μM, and 1.4 mg/mL fatty acid-free BSA in siliconized glass tubes. Bound ligand was separated from free ligand by vacuum filtration. Non-specific binding was determined using 10 μM GTPS. Basal binding was assayed in the absence of the ligand and in the presence of GDP.

Tissue levels of antagonists: Mice received a single dose (Compound 8: 3 to 30 mg/kg ip and Compound 4: RR/SR 10-50 mg/kg) or rimonabant and were sacrificed 1 hour later. Blood was collected, and the mice were perfused with phosphate buffered saline for 1 min to remove drug from the intravascular space before removing the brain and liver. Drug levels in tissue homogenates and plasma were determined by using LC-MS/MS.

Locomotor Activity: Locomotor activity was quantified by the number of disruptions of infrared XYZ beam arrays with a beam spacing of 0.25 cm in the Promethion High-Definition Behavioral Phenotyping System (Sable Instruments, Inc., Las Vegas, Nev., USA).

### Results

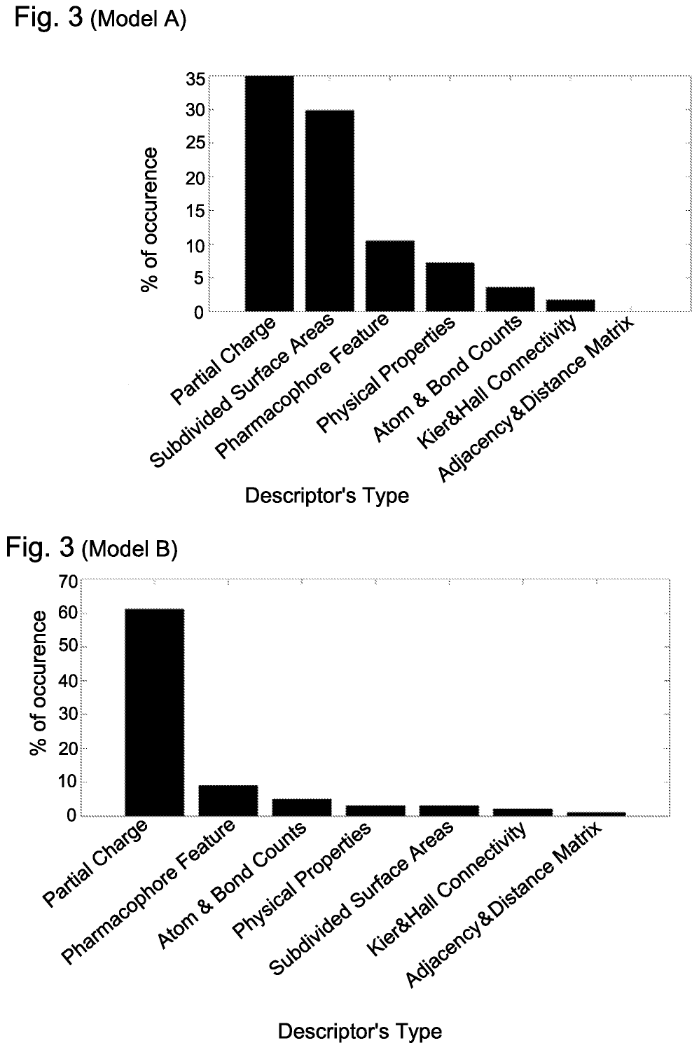

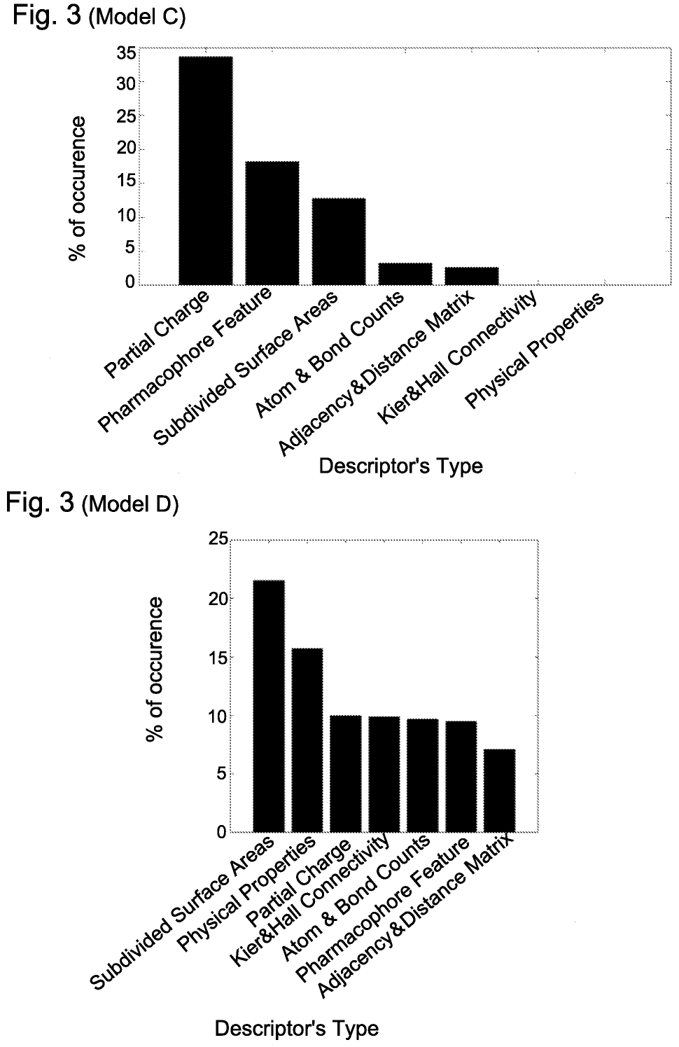

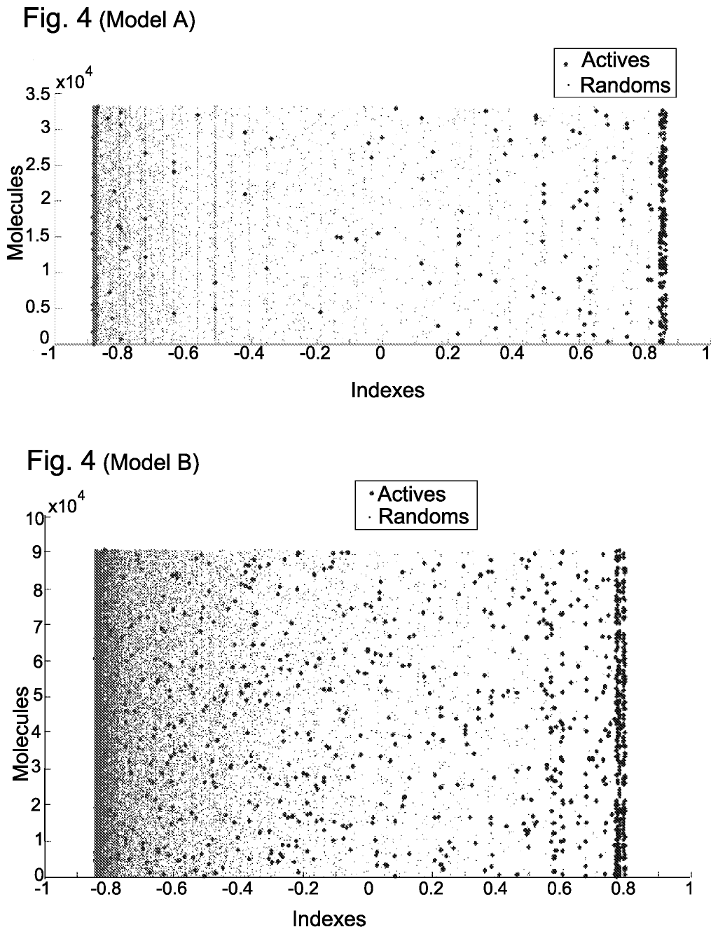

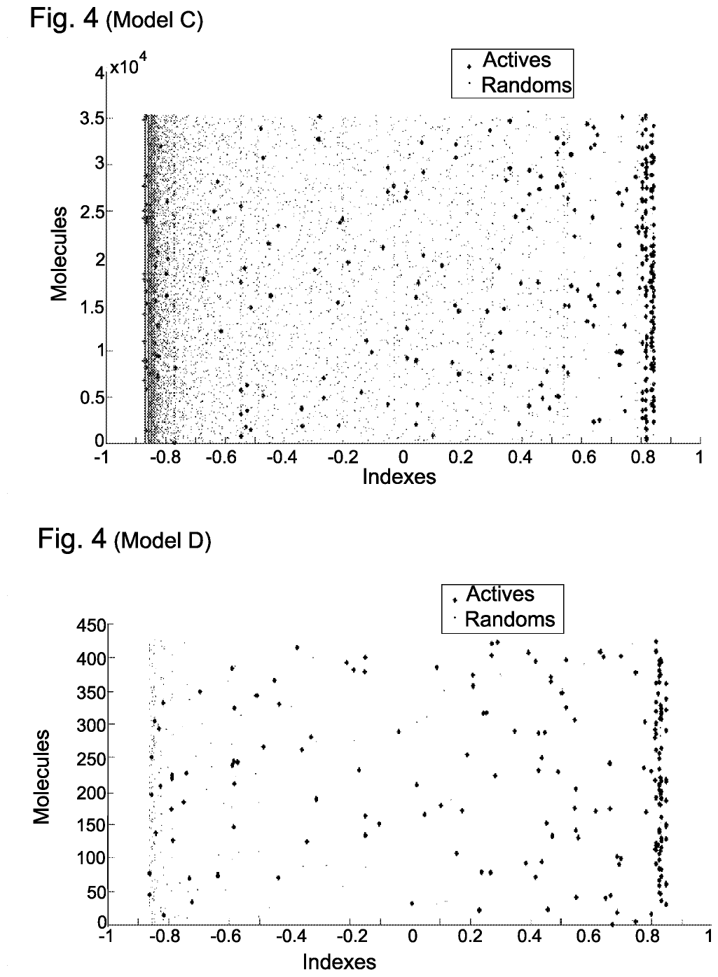

ISE activity models: Several Models were built by ISE for CB1R antagonist activity, and four were selected for VS. The models contained filters with five ranges of descriptors each; the models differed from each other by the number and composition of filters (for detailed occurrences of the descriptors in the different models; FIG. 3). All the models had quite similar quality, as it was evident from MCC values for the top filter, from the mean MCC in each model and from the high AUC (>0.9) of each. These numbers, taken together, indicated successful classifications by all four models (Table 4). Each of the actives and the inactives got an index for its success in each of the models. Scatter plots and tables for the learning set in each model helped to determine how to analyze results for the subsequent VS of millions of molecules. A cutoff index for VS in each model was required for deciding that molecules above that index would be further examined as potential hits. Scatter plots of the internal test sets distribution in each model are shown in FIG. 4. We chose a different cutoff for each model based on the TP/FP rate, the larger, is the better. We present those cutoffs (lowest line) together with results for the external set (Table 5).

TABLE 4
Parameters of the different models.

| | Model | Model | Model | Model |
|---|---|---|---|---|
| ISE models | A | B* | C | D |
| Number of filters | 1399 | 1895 | 1960 | 995 |
| MCC of the top filter | 0.78 | 0.72 | 0.75 | 0.75 |
| Mean MCC | 0.75 | 0.67^{x} | 0.7 | 0.69 |
| AUC | 0.91 | 0.95 | 0.91 | 0.92 |
| EF | 60* | 94** | 82* | 2* |
| Sensitivity | 0.54* | 0.37** | 0.42* | 0.42* |
| Specificity | 0.99* | 0.99** | 0.99* | 0.97* |
| TP/FP | 1.15* | 16** | 3.5* | 16.2* |

×Only top 1000 filters used for screening.
*Above index 0.8,
**above index 0.7.
MCC—Mathew correlation coefficient,
AUC—area under the ROC curve,
EF—Enrichment factor.

Test set screening: We screened the external test set of active and inactive molecules collected from CHEMBL database (2970 molecules) through the four models (Table 5).

TABLE 5
External test set validation results.

| ISE models | Model A | Model B | Model C | Model D |
|---|---|---|---|---|
| AUC | 0.76 | 0.87 | 0.84 | 0.82 |
| EF | 1.41 | 1.41 | 1.38 | 1.41 |
| Sensitivity | 0.06 | 0.09 | 0.08 | 0.02 |
| Specificity | 1 | 1 | 0.99 | 1 |
| TP/FP | ∞ | ∞ | 42.2 | ∞ |
| Index cutoff | 0.779 | 0.764 | 0.796 | 0.8 |
| used for VS | | | | |

Screening the Enamine database through the four models: The Enamine database (2,170,859 molecules) was screened through each of the four models. The results are summarized in Table 6. Different numbers of hits (above the cutoff index of each, line 2) were found for each of the models. Combining them gave 626 hits, which were reduced to 498 after removing duplicates.

TABLE 6
Screening results of the Enamine database through the four models.

| Models | Model A | Model B | Model C | Model D |
|---|---|---|---|---|
| Number of hits | 238 | 237 | 13 | 138 |
| Index cutoff for VS | 0.779 | 0.764 | 0.796 | 0.8 |

| | |
|---|---|
| Total hits | Total = 498 unique hits |

Applying the criteria for peripheral action: The Enamine database (2,170,859 molecules) was screened through each of the four models. The results are summarized in Table 6. Different numbers of hits (above the cutoff index of each, line 2) were found for each of the models. Combining them gave 626 hits, which were reduced to 498 after removing duplicates.

CB1R binding: The actual affinity of the 14 candidates was tested in a CB1R competitive binding assay. Table 7 lists the molecular structures and their binding assay results. Ten compounds showed good affinity for the CB1R. The most potent compounds, Compound 8 and Compound 10, had a CB1R Kᵢ of ˜400 nM. Moreover, rimonabant was tested under the same conditions, and its Kᵢ values for the CB1R was 4.7 nM, in line with our previously reported values.

TABLE 71
Chemical properties of the VS novel compounds.

| | | | | | Hydrogen | | |
|---|---|---|---|---|---|---|---|
| | ISE | Index | | | bond | | |
| Compound | model | Score | MW | cLogP | (donor) | PSA | Ki (μM) |
| Compound 1 | Model | 0.853 | 480.39 | 6.1 | 3 | 73.98 | 0.0 |
| | A (D) | (0.829) | | | | | |
| Compound 2 | Model | 0.853 | 483.51 | 5.5 | 3 | 94.4 | 2.1 |
| | A (B) | (0.791) | | | | | |
| Compound 3 | Model | 0.812 | 524.01 | 4.5 | 3 | 108.13 | 1.8 |
| | D | | | | | | |
| Compound 4 | Model | 0.806 | 463.9 | 4.9 | 3 | 87.1 | 1.1 |
| | A | | | | | | |
| Compound 5 | Model | 0.804 | 469.4 | 4.28 | 3 | 87.3 | 0.0 |
| | A | | | | | | |
| Compound 6 | Model | 0.791 | 502.4 | 5.78 | 3 | 91.0 | 1.0 |
| | B | | | | | | |
| Compound 7 | Model | 0.804 | 460.9 | 4.9 | 4 | 86.0 | 3.9 |
| | D | | | | | | |
| Compound 8 | Model | 0.853 | 497.5 | 5.6 | 3 | 94.4 | 0.408 |
| | A | | | | | | |
| Compound 9 | Model | 0.791 | 484.4 | 6.4 | 3 | 78.9 | 0.0 |
| | B | | | | | | |
| Compound 10 | Model | 0.853 | 499.4 | 6.8 | 0 | 84.2 | 0.414 |
| | A (B) | (0.791) | | | | | |
| Compound 11 | Model | 0.847 | 496.4 | 6.1 | 3 | 82.0 | 1.3 |
| | A | | | | | | |
| Compound 12 | Model | 0.791 | 474.0 | 5.9 | 3 | 102.3 | 3.4 |
| | B | | | | | | |
| Compound 13 | Model | 0.791 | 482.0 | 5.1 | 3 | 91.0 | 6.5 |
| | B (D) | (0.8) | | | | | |
| Compound 14 | Model | 0.812 | 474.0 | 5.4 | 3 | 73.9 | 0.938 |
| | A (D) | (0.821) | | | | | |

Testing the activity of the selected compounds: By using [³⁵S]GTPγS binding assay, we next evaluated the activity (agonism, antagonism, inverse agonism) properties of 8 out the 14 molecules that showed the highest affinity for the CB1R. The test was performed for each compound with and without the CB1R agonist HU-210 (100 nM). Whereas two compounds (Compound 3 and Compound 4) showed neutral antagonism properties, five others—Compound 2, Compound 8, Compound 11, Compound 12, and Compound 14 were defined as inverse agonists (Table 8). While Compound 10 could be a positive allosteric modulator (PAM).

The values of the affinity and selectivity for CB1R of the 14 compounds are summarized in Table 7. The most potent compounds were examined in GTPγS binding in mouse brain membranes (Table 8), and whether they were able to ameliorate the stimulatory action of the potent CB1R agonist HU-210 (Table 8), suggesting that some of the compounds are pure antagonists and others are inverse agonists.

Next, we examined the enantio-selectivity effect of the most potent compounds that have chiral center (Compound 3, Compound 4, Compound 5, Compound 9, Compound 14). The configuration labeling (R, S, RR, SR, etc.) does not represent the real configuration for now. Some compounds showed differences in their ability to bind the receptor (Table 9).

TABLE 9
The ability of each compound, separated
to diasteriomers, to bind the CBIR.

| | Ki (μM) | | |
|---|---|---|---|
| Compound | Mix | R | S |
| Compound 2 | 3.36 | 4.00 | 6.33 |
| Compound 3 | 1.1 | 1.3 | 1.3 |
| Compound 8 | 0.346 | 0.290 | 0.977 |

| | Ki (μM) | | | | |
|---|---|---|---|---|---|
| Compound | Mix | RR | SS | RS | SR |
| Compound 4 | 1.0 | 0.54 | 1.66 | 8.46 | 0.48 |
| Compound 14 | 1.2 | 1.1 | 1.8 | 5.6 | 5.5 |

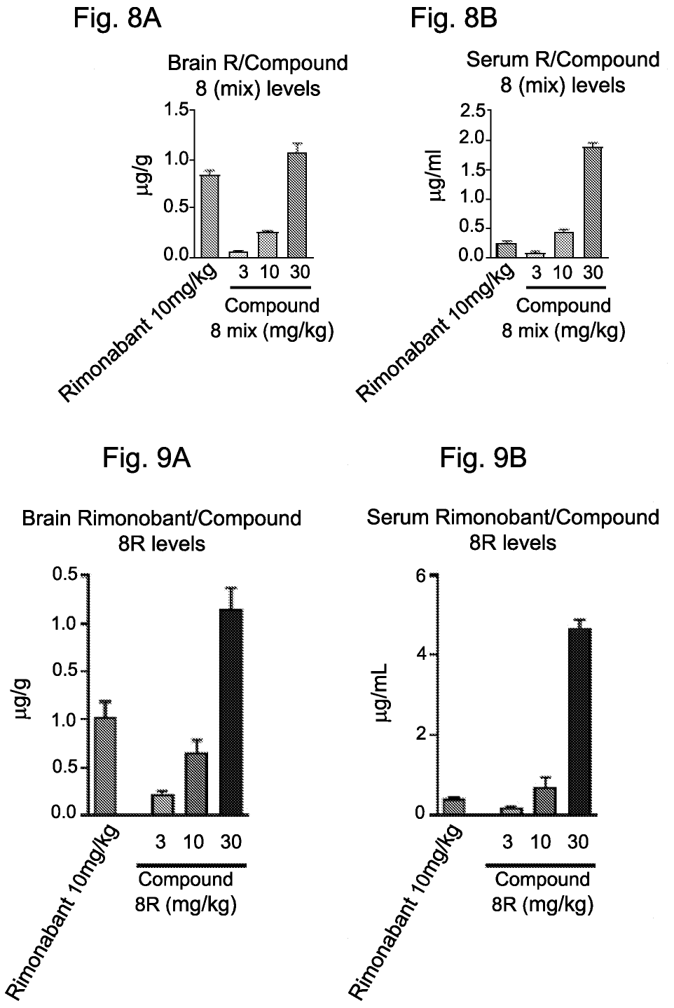

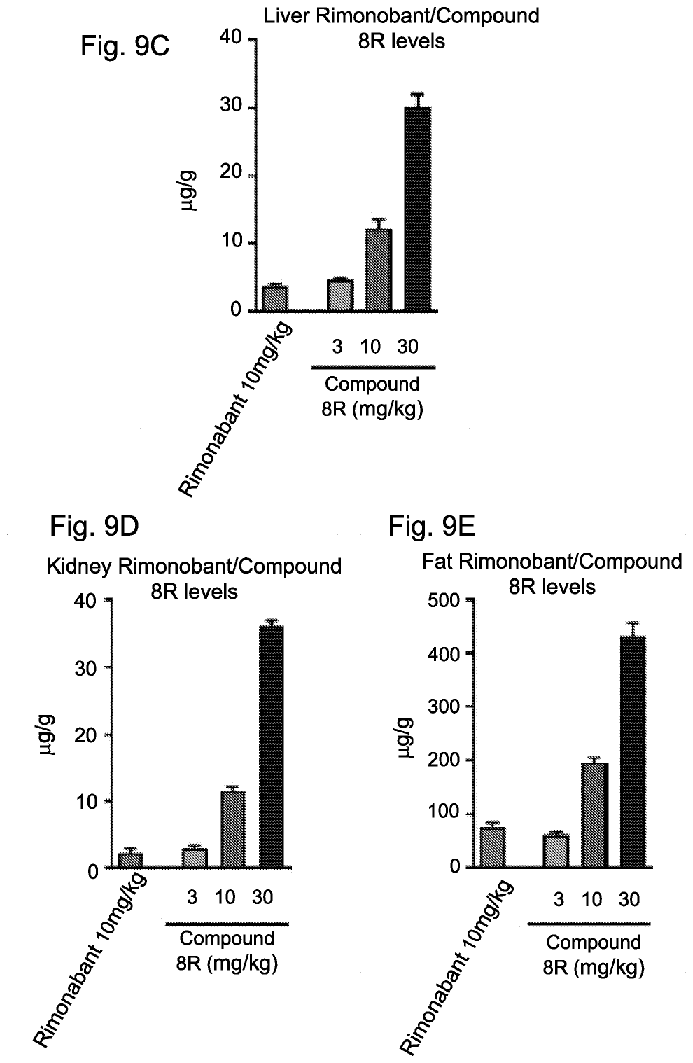

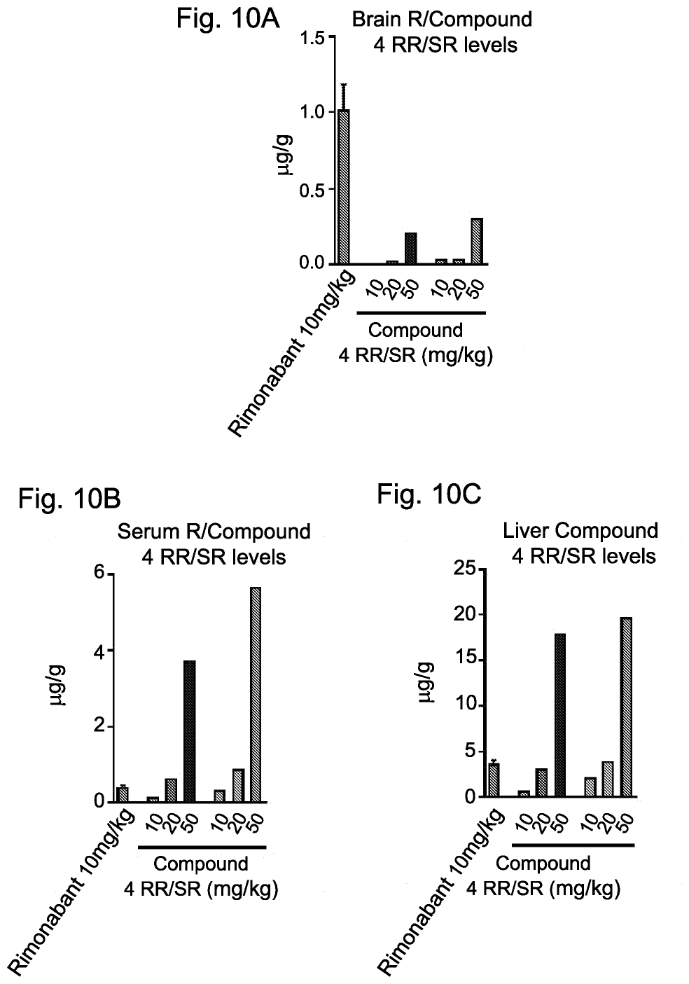

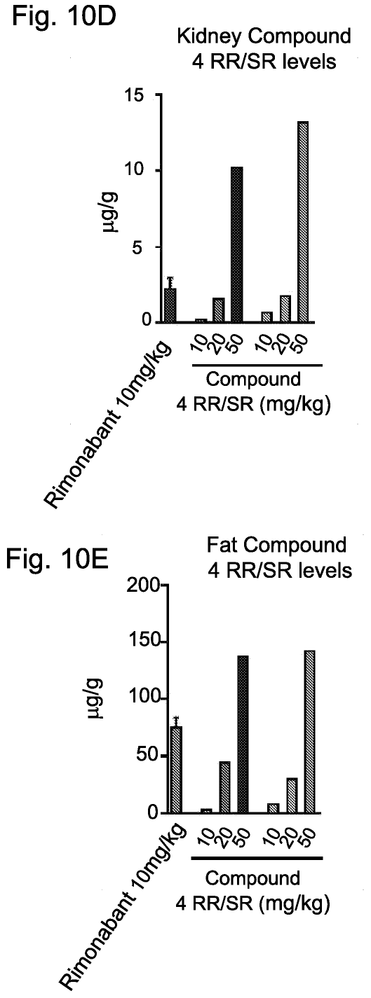

Importantly, the most potent compounds (Compound 8R mix, and Compound 4 RR and SR) displayed markedly reduced brain penetrance, as reflected by their reduced brain levels and increased serum levels following an administration of the compounds at different doses (3, 10 and 30 mg/kg, ip; FIGS. 8A, B for Compound 8 mix and 10, 20, 50 mg/kg, ip; for Compound 4-RR and Compound 4-SR FIGS. 9A-D).

We next tested whether the reduced brain penetrance of Compound 8/Compound 4 is associated with an attenuation of behavioral effects. To that end, we compared the effects of Compound 8-R and Compound 4-RR/Compound 4-SR and rimonabant in inducing CB1R-mediated hyperactivity.

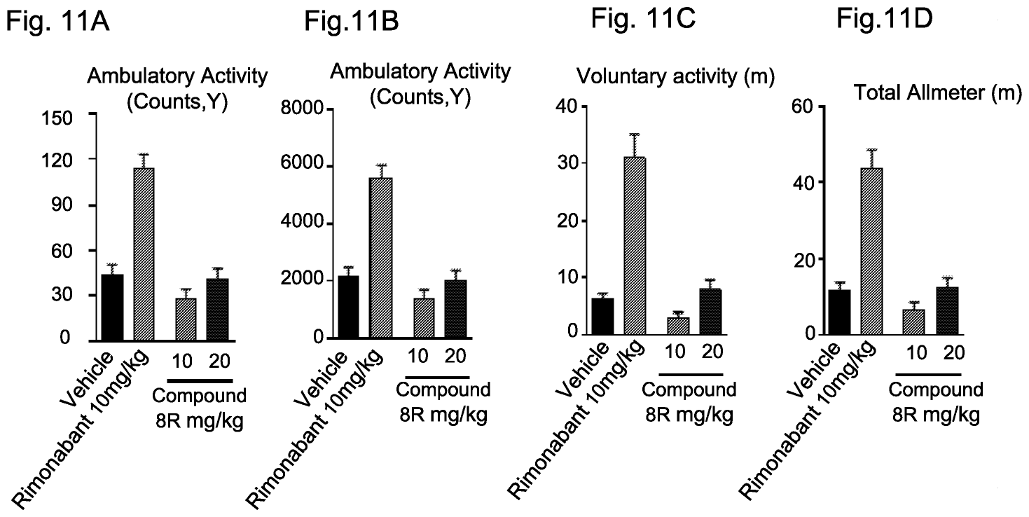

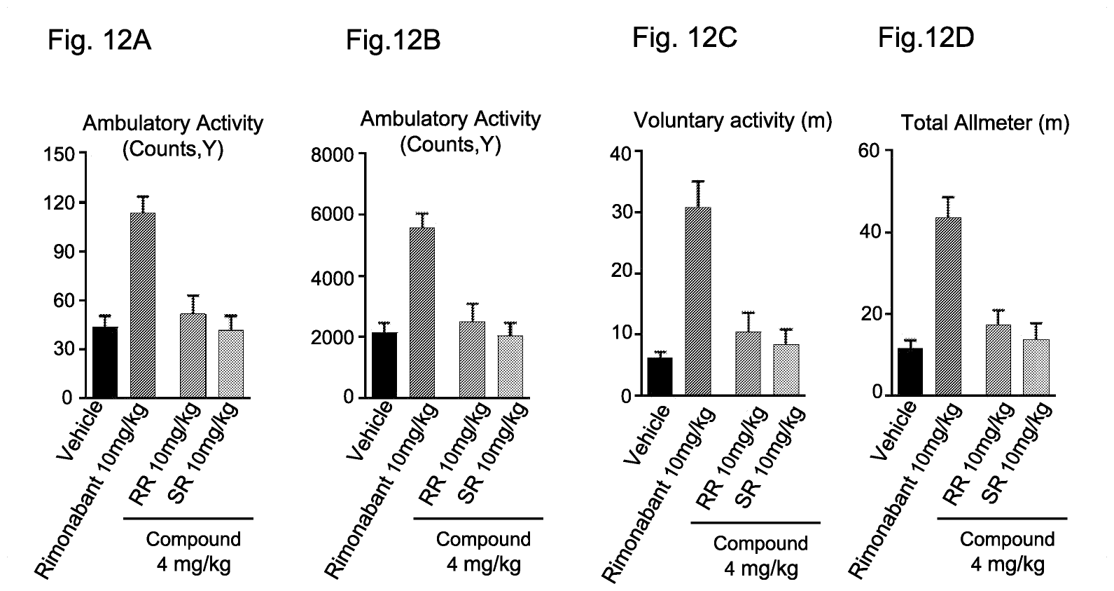

Rimonabant (10 mg/kg, ip), but not Compound 8-R (10 and 20 mg/kg, ip) and Compound 4-RR/Compound 4-SR (10 mg/kg, ip), induced a marked increase in the activity profile in mice (FIGS. 11 and 12).

Next, we assessed the binding and nature of activity of 8 compounds against the CB2R. The values of the affinity for CB2R of the 8 compounds are summarized in Table 10. Each compound was then examined for GTPγS binding in human cell membranes in order to define their activity profile (agonist, antagonist, inverse agonist; Table 10).

The ability of Compound 4 and Compound 8 to bind to the CB2R was further assessed after separating the racemic mixture into isolated enantiomers. Compound 4 has two chiral centers, resulting in 4 enantiomers, whereas Compound 8 has one chiral center, resulting in 2 enantiomers. The configuration labeling (R, S, RR, SR, etc.) does not represent the real configuration for now. Some compounds showed differences in their ability to bind the CB2R as documented in Table 11.

The success of peripherally restricted CB1R antagonists to reduce obesity, reverse leptin resistance and improve hepatic steatosis, dyslipidemia and insulin resistance in genetically and diet-induced obese mice indicates that there is no need to block central CB1Rs for the treatment of metabolic disorders. The increasing interest in finding novel peripherally restricted CB1R antagonists, led us to look for new candidates. Our ISE algorithm has already demonstrated an ability to discover novel scaffolds while learning from other scaffolds. We applied ISE to build activity models for CB1R antagonists. The models that got the best classification performance were used for VS. The active molecules used to build the four models (IC₅₀†Kᵢ) differed in the ranges of molecular weight and LogP (descriptors calculated by MOE).

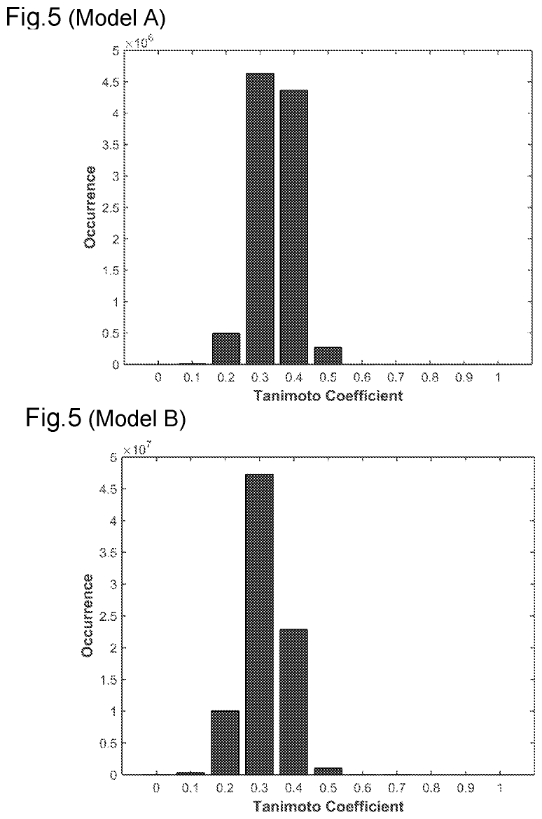

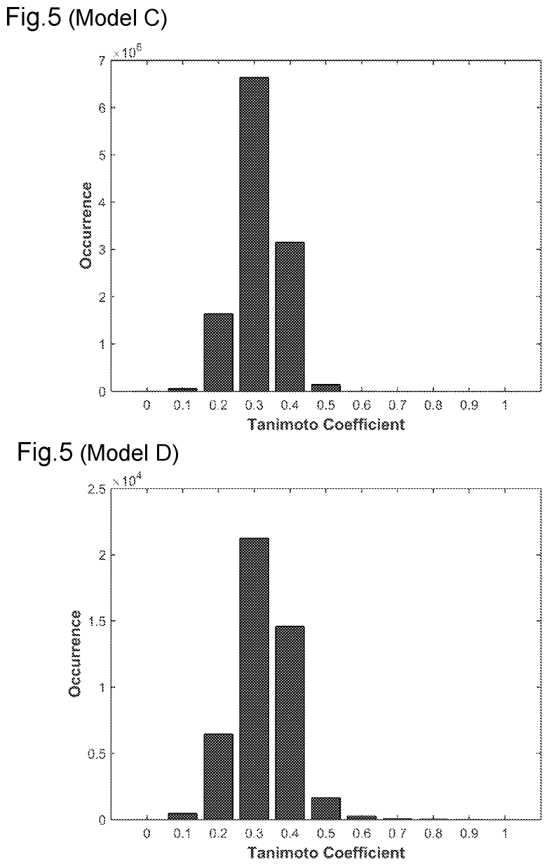

Preferred model: The models were validated twice—initially by five cross-validation (for model construction) and subsequently by an external validation set (on the full model). Despite the different numbers of learning set molecules in the four models—the classification performance is quite similar in all four, with MCC ˜0.7 and AUC>0.9. The Enrichment factor values, which are large for models A, B and C, is much smaller for model D (high vs. low actives). This is a simple result of the EF equation (Eq. 2). Due to the fact that the number of positives is just about half of the total, and the TP (above an index of 0.8 in that model) equals 16.2 FP, the numerator is nearly 1 (16.2FP/17.2FP) and so the result for EF is ˜2. The average Tanimoto between the active and random molecules used to build the different models is 0.39, 0.36, 0.36 and 0.37 for models A-D respectively (see Tanimoto distribution in FIG. 5). Thus, the learning sets are all highly diverse and have similar model parameters except for the EF (Table 4).

$$Equation 2. Enrichment Factor .$$

$$EF = \frac{TP / ( TP + FP )}{( {N}_{POSITIVES} / {N}_{Total} )}$$

There are some differences among the models in the proportion of the “Partial charge” descriptors, which are the most abundant in Models A-C. In Model A and C, where the decoys are from the ZINC database they comprise ˜30% of the descriptors, in Model B where the decoys are from the Enamine database it is ˜60%, and in Model D where we used only active molecules, this descriptors family contributes only 10%. The next representative family is the “Pharmacophore Feature” descriptors, ˜10% in Models A-D, which are set to: Donor, Acceptor, Polar (both Donor and Acceptor), Positive (base), Negative (acid), Hydrophobic and Others. In model A and C, we find a contribution of the “Subdivided Surface Area” descriptors based on an approximate accessible van der Waals surface area (in Å²) calculation for each atom, vi along with some other atomic property, pᵢ. (FIG. 3).

External vs. internal test sets: The external test set (number of active molecules-2098, number of inactive molecules-872), got smaller AUC values than the internal test set (with 5-fold cross validation), but it is still high enough (AUC ˜0.8), indicating a none-random classifier. The EF was nearly similar (˜1.4) for all the external set screenings through the four models, again being lower than the EF values of all 4 models above. These findings exhibit a drop in performance that is experienced during external validation, for situations when the tested compounds are distinct from the training set. Average Tanimoto values of 0.34, 0.33, 0.34, 0.33 for the external set were found for comparing with the active molecules of models A-D respectively. An average Tanimoto Coefficient of 0.3 between the external test set and our training set provides a possible explanation for the lower performance of the external test—as the molecules are highly different in both sets. There was a large portion of FN, however the number of FP was zero for all models, except for Model C with only four inactive molecules that had been predicted as actives. However, the four properties used to determine the APD of the learning set (FIG. 2) differ for the test set—not in the ranges, but in the distribution of the values, that is much narrower than the learning sets. The distribution of the other descriptors could be the reason for the decline in the performance in this case.

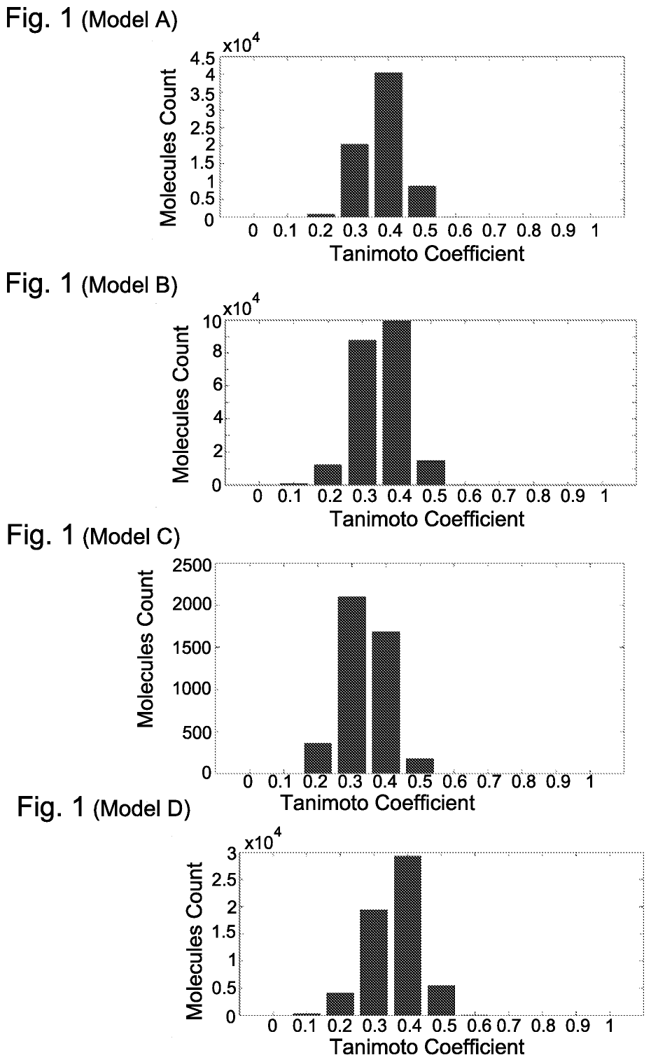

Diversity of the resulting hits: Screening the Enamine database (2,170,859 molecules) through the four models and combining the results yielded in total ˜500 hits (some hits appear in more than one model, Table 7). The diversity is measured between the discovered virtual hits and the original active ones used to produce the models. In Model A, we compared the 238 hits with the 296 actives used to build the model, and only two hits were found to have a Tanimoto value above 0.7, which indicates a highly diverse set. In Model B, 237 molecules (with index above 0.764) were compared to 906 known actives, the highest Tanimoto value was 0.75, but only five hits got a Tanimoto index greater than 0.7. Considering a cutoff of 0.796, we got only 13 hits in Model C, all of them have a Tanimoto value lower than 0.7, with a maximum value of 0.58. In the last Model, if we compare the 138 hits to the whole set (highly and low active molecules-425 molecules) the highest Tanimoto is 0.65 (FIG. 1). The obvious conclusion is that all the predicted hits are different than the molecules we learned from, that were the basis for discovering those hits. This is a unique feature of ISE, which is a result of ignoring structures and focusing on properties.

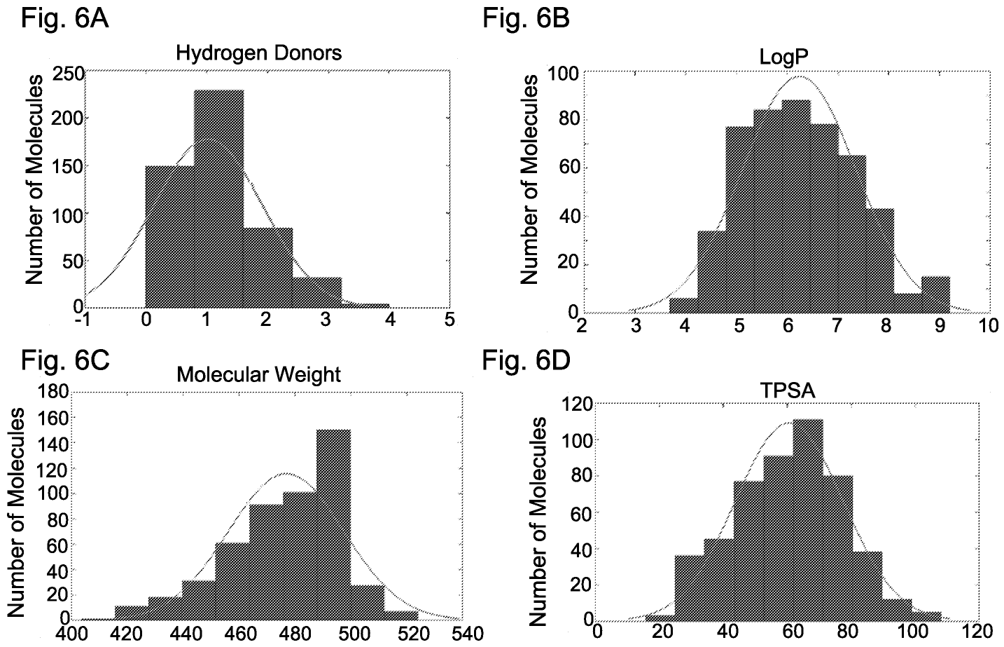

Peripheral filtration: BBB penetration may be a liability for many of the non-CNS drug targets, and a clear understanding of the physicochemical and structural differences between CNS and non-CNS drugs may assist both research areas. Molecular weight plays a crucial role for CNS penetration and drug bioavailability in general. For CNS acting drugs, the mean value of MW is 310 compared with a mean MW of 377 for all marketed orally active drugs. Increasing lipophilicity increases brain penetration. The mean value for cLogP for the marketed CNS acting drugs is 2.8. Another parameter used for BBB penetration prediction is the polar surface area (PSA), which is significantly less for CNS drugs (2-64 Å²) than for non-CNS oral drugs (89-185 Å²). Number of H-bond Donors (HBD) ranges between 0-2 and the number of H-bond acceptors ranges between 2-8 for CNS drugs. Peripheral filtration according to these physicochemical properties (Table 3) left us with 33 molecules only, some were enantiomers, and 15 were purchasable. The prediction for these molecules of logBB and of CNS entry (on the Enamine website) is zero for all, meaning that they are indeed not expected to enter the CNS. FIG. 6 shows the distribution of peripheral properties for the ˜500 hits from VS. The most crucial factor that determined the peripheral expected activity is the number of HBD, which should be greater/equal to three. After applying this criterion 36 molecules remained (Table 12). We add another 3 molecules that does not fit this criterion to increase the number for testing in vitro.

TABLE 12
The number left out of the 498 hits after applying each criterion.

| Criteria | Number of molecules fulfil the criteria |
|---|---|
| HBD ≥3 | 36 |
| Molecular weight ≥450 | 442 |
| PSA ≥70 | 139 |
| LogP ≥4 | 494 |

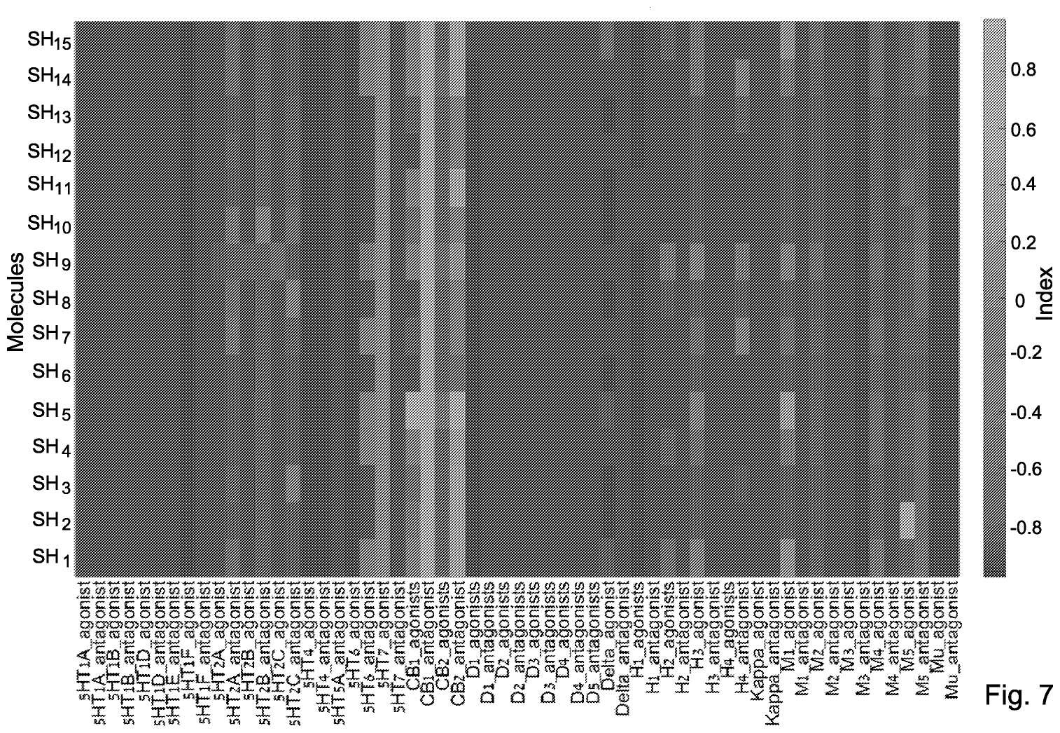

Diversity of the 15 hits to the learning set: Comparing the similarity of the 15 hits to 906 known actives from Chembl, we found a maximum Tanimoto coefficient value of 0.61 and an average of 0.41. A higher average than what was found between the different learning sets. The SEA algorithm, based on ligand similarity to known actives of targets, does not detect any probability of our final candidates to hit CB1R. The candidates for peripheral CB1R antagonism were examined by a large set of ISE models for GPCRs activity (Serotonin, Histamine, Muscarinic, Opioid, Dopamine and Cannabinoid agonists and antagonist): considering an index cutoff of 0.7, no molecule passes that cutoff, except for Compound 4 that got an index of 0.891 in the CB1R agonist model. See supporting information in FIG. 7.

Binding tests for the 14 candidates: In the binding assay, 10 candidates out of the 14 showed a good affinity for CB1R (Kᵢ=0.408-6.3 μM), compared to 4.7 nM of rimonabant. Compound 8 and Compound 10 had the greatest affinity values ˜400 nM (Table 7). As to activity—Compound 3 and Compound 4 showed antagonist properties, while Compound 2, Compound 3, Compound 8, Compound 11, Compound 12, and Compound 14 were defined “inverse agonists”, as they decreased the basal [³⁵S]GTPγS binding under 100%, when adding HU210, a synthetic agonist. While Compound 10 could be a positive allosteric modulator (PAM), as it decreases the basal binding of [³⁵S]GTPγS, but when added with HU210, it increases its activity.

Predictions and proof: The cutoff for VS is determined by the EF and TP/FP rate of each model. The enrichment is used to identify active molecules for the target of interest when compared with random selection, and the top scoring molecules are prioritized for ongoing into experimental testing, which is our cost-effective strategy in drug discovery programs. Compounds that are genuinely active against the target are rare (˜0.01-0.1% of library), and are easily masked by a high incidence of false-positives in a screen. In a “natural” distribution, the number of active compounds would be much smaller than the number of inactive compounds for each particular activity, thus we diluted the active molecules with random molecules when building the models, based on APD of four properties, to keep the learning set at a range of chemical structures for which the model is considered to be relevant and which makes the classification more challenging. To mimic a reported proportion of hit discovery in HTS, of 1:1000, we reduced that proportion in VS to a 1:100 dilution of actives by inactives in order to save time. Once a model was constructed, we needed to reduce the values of TP/FP ten-fold, and by that obtaining numbers that are more relevant to reality.

If we consider each model alone, ignoring the common molecules that appear in more than one model as shown in Table 7, for model A the TP/FP rate is 0.12, instead of the original 1.2, meaning that out of 112 molecules, 12 are expected statistically to be active. Eight candidates out of the 15 molecules sent for experiment were from model A, representing a chance of finding at most one hit from that model, six found to have affinities (0.408-1.1 μM). For models B and D, the rate is 1.6, so out of 26 molecules, 16 are expected to be active. Model B supplied 6 candidates (3 commons with other models), of which five show binding affinity (0.414-6.5 μM), and the five of model D, four showed binding affinity (1.8-6.5 μM), for model C only one molecule pass the peripheral criteria but it was untested because of stability issues. Molecules sent for experiments included the criteria of peripheral selectivity, while the total numbers of CB1R antagonist candidates is much larger. The expected hits out of 238 proper candidates of Model A is ˜25. For the 237 candidates of model B it is much larger, close to 150. Model C could supply only ˜3 out of the 13 candidates, and model D could supply ˜85 out of its 138 candidates, overall some 250 molecules.

The extent to which the random set affects the VS: The concept for the applicability domain of a model is related to the term model validation, is the model within its domain of applicability possesses a satisfactory range of accuracy within the intended application of the model and will be applied with good predictive performance? Another interpretation for APD is “the group of chemicals for which the model is valid or with the highest predictive performance”. Comparing models A and B, both are based on IC₅₀ data of actives, but the random molecules are chosen from two different databases of ZINC and ENAMINE. The VS were performed for the ENAMINE database, we got similar number of hits (238 and 237 for model A and B), and 80 molecules were common between the two models. If we look at the tested hits; 6 molecules out of the 8 from model A have measured Ki affinity, similarly 5 molecules out of the 6 from model B have affinity. The two common molecules (from A and B models) got a Ki of 2.1 and 0.414 μM. Therefore, there seems to be no difference between using randoms from ZINC or from ENAMINE when it gets to VS of the ENAMINE database.

Ki or IC₅₀ for building models: The type of activity measurement, biological data accuracy and experimental uncertainty affect the prediction performances and interpretation of computational models built for that data set. Binding affinity provides information on the strength of the interaction between a drug-target association and it is usually expressed in measures such as dissociation constant (K_{d}), inhibition constant (Ki). The smaller the K_{d} value, the greater the binding affinity of the ligand for its target, similarly the low IC₅₀ values the more potent the ligand is towards its target. HTS libraries are usually evaluated on larger numbers of different targets and confirmatory screening assays typically produce IC₅₀ values, this is why IC₅₀ measurements are expected to cover target space more broadly than Ki values, which are more costly than approximate activity measurements and often only carried out for small numbers of high-interest targets. Model A, generated from an IC₅₀ dataset, while model C from Ki dataset, both with high activity measurements. From model A we got 238 hits, and six showed affinities with Ki=0.408-1.1 μM, while model C yielded only 13 hits. The most potent compounds that were tested and reported in this paper are undergoing evaluation in mice for their distribution between the brain and the periphery.

1. A compound for use in medicine, the compound being selected from

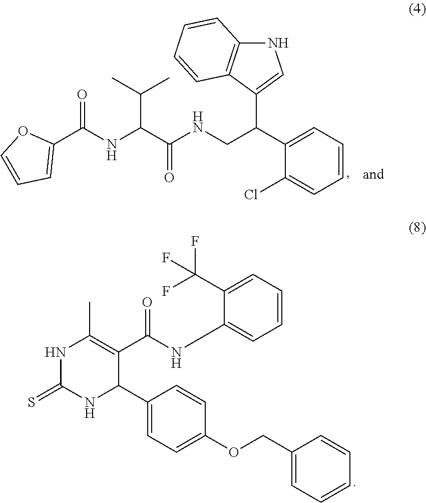 [SMILES: CC(C)C(NC(=O)c1ccco1)C(=O)NCC(c1ccccc1Cl)c1c[nH]c2ccccc12 ; CC1=C(C(=O)Nc2ccccc2C(F)(F)F)C(c2ccc(OCc3ccccc3)cc2)NC(=S)N1]

2. A compound for use in medicine, the compound being selected from:

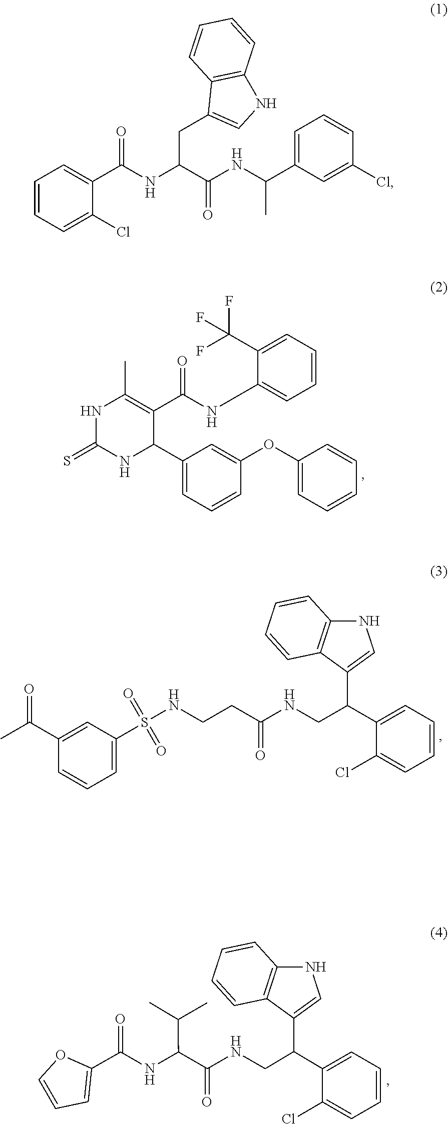 [SMILES: CC(NC(=O)C(Cc1c[nH]c2ccccc12)NC(=O)c1ccccc1Cl)c1cccc(Cl)c1 ; CC1=C(C(=O)Nc2ccccc2C(F)(F)F)C(c2cccc(Oc3ccccc3)c2)NC(=S)N1 ; CC(=O)c1cccc(S(=O)(=O)NCCC(=O)NCC(c2ccccc2Cl)c2c[nH]c3ccccc23)c1 ; CC(C)C(NC(=O)c1ccco1)C(=O)NCC(c1ccccc1Cl)c1c[nH]c2ccccc12]

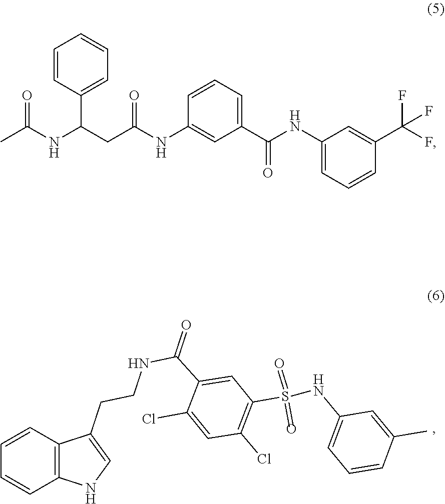

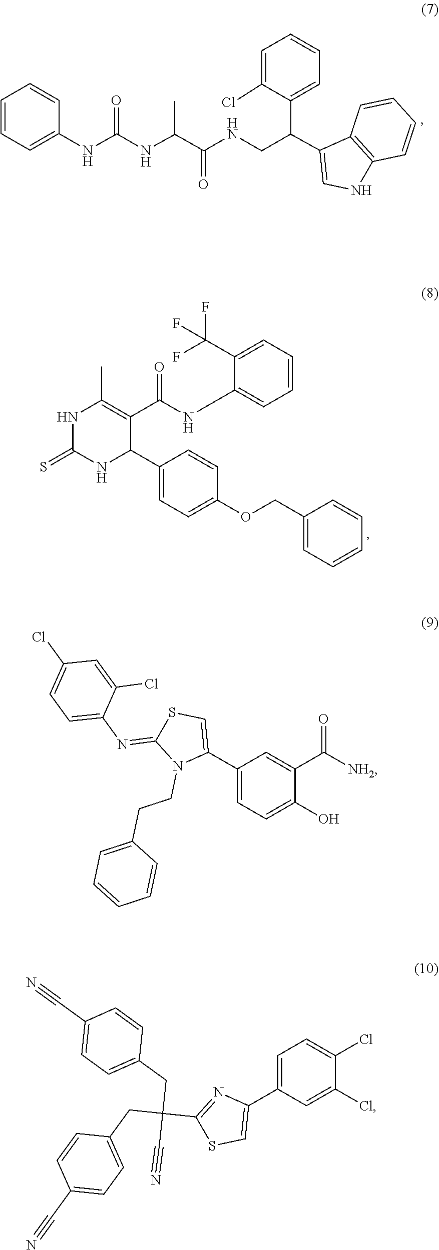 [SMILES: CC(NC(=O)Nc1ccccc1)C(=O)NCC(c1ccccc1Cl)c1c[nH]c2ccccc12 ; CC1=C(C(=O)Nc2ccccc2C(F)(F)F)C(c2ccc(OCc3ccccc3)cc2)NC(=S)N1 ; NC(=O)c1cc(-c2cs/c(=N\c3ccc(Cl)cc3Cl)n2CCc2ccccc2)ccc1O ; N#Cc1ccc(CC(C#N)(Cc2ccc(C#N)cc2)c2nc(-c3ccc(Cl)c(Cl)c3)cs2)cc1]

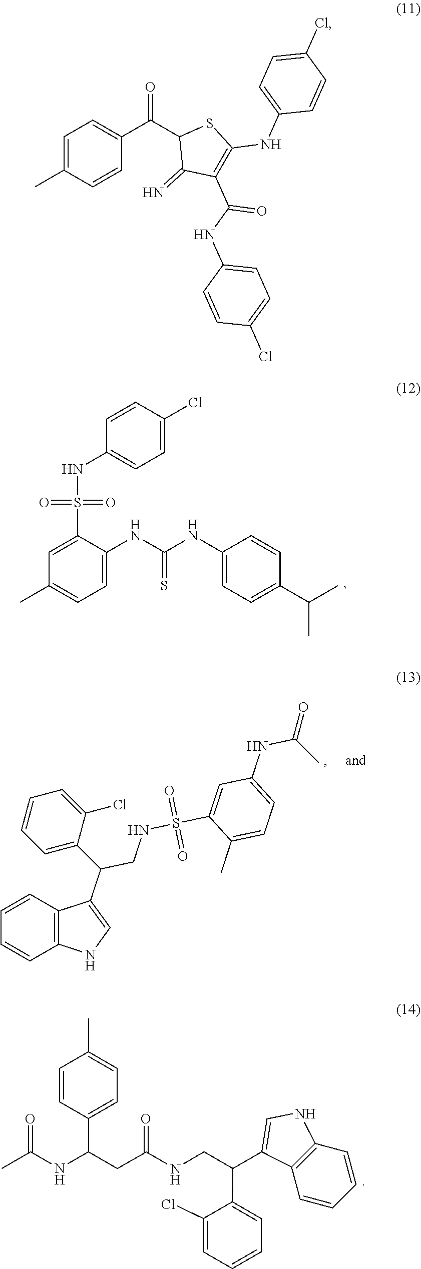

3. The compound according to claim 2, being a compound herein designated compound (4) or compound (8).

4. A compound of the general formula (I) or (II) for use in medicine: 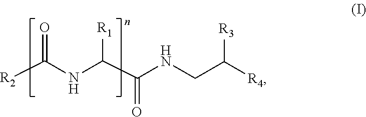
wherein in a compound of formula (I):
n is an integer between 1 and 3;
R1 is a C1-C5alkyl; and
each of R2, R3 and R4, independently of the other is a C6-C10aryl, a C5-C10heteroaryl or a C5-C10carbocycle;
or 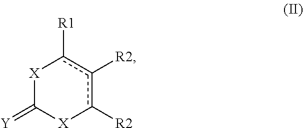
wherein in a compound of formula (II):
each X is a heteroatom selected from O, NH and S;
Y is a heteroatom selected from O, NH and S;
R1 is a C1-C5alkyl;
R2 is a —(C═O)NH—R3;
R3 is a C6-C10aryl or a C5-C10heteroaryl; and
one of the bonds designated [symbol] is a double bond and the other is a single bond.

5. The compound according to claim 1, for use in preventing or treating a metabolic syndrome or disorder.

6. The compound according to claim 5, wherein the metabolic syndrome or disorder is selected from obesity, insulin resistance, diabetes, coronary heart disease, liver cirrhosis, dyslipidaemia, hypertension, chronic inflammation, a hypercoagulable state, and chronic kidney disease.

7. The compound according to claim 1, for use in a method for treating a subject to reduce body fat or body weight, or to treat insulin resistance, or to treat diabetes, or to reduce or control high blood pressure, or to improve a poor lipid profile with elevated LDL cholesterol, low HDL cholesterol, and elevated triglycerides, or to treat acute and chronic kidney injury, or to treat a metabolic syndrome.

8-13. (canceled)

14. A pharmaceutical composition comprising a compound designated compound (1) through compound (14) 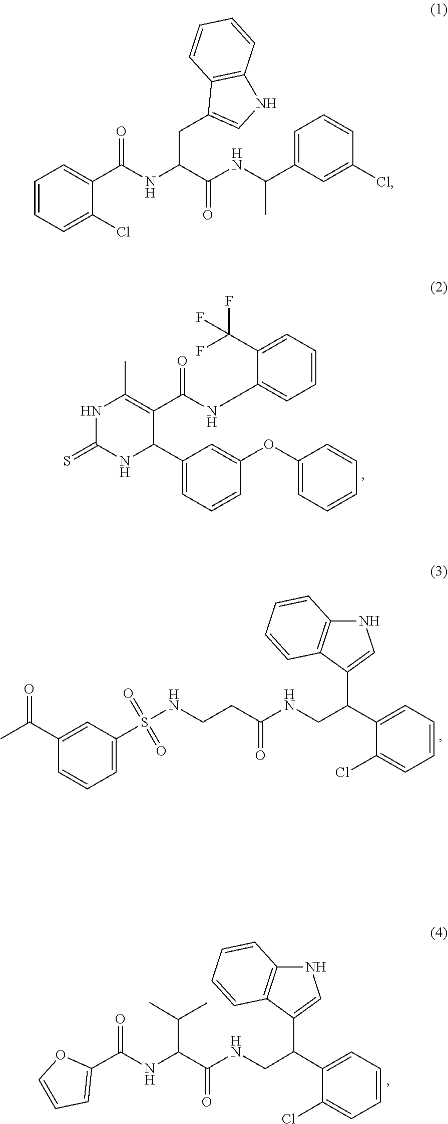 [SMILES: CC(NC(=O)C(Cc1c[nH]c2ccccc12)NC(=O)c1ccccc1Cl)c1cccc(Cl)c1 ; CC1=C(C(=O)Nc2ccccc2C(F)(F)F)C(c2cccc(Oc3ccccc3)c2)NC(=S)N1 ; CC(=O)c1cccc(S(=O)(=O)NCCC(=O)NCC(c2ccccc2Cl)c2c[nH]c3ccccc23)c1 ; CC(C)C(NC(=O)c1ccco1)C(=O)NCC(c1ccccc1Cl)c1c[nH]c2ccccc12] 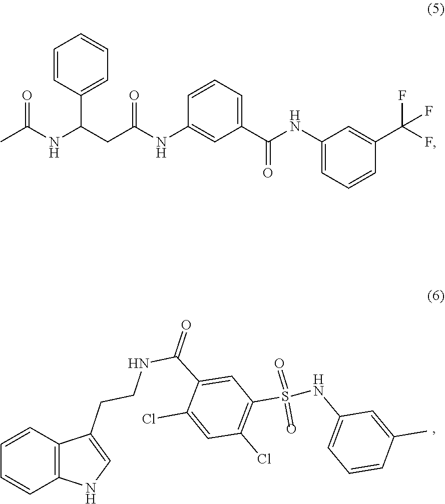 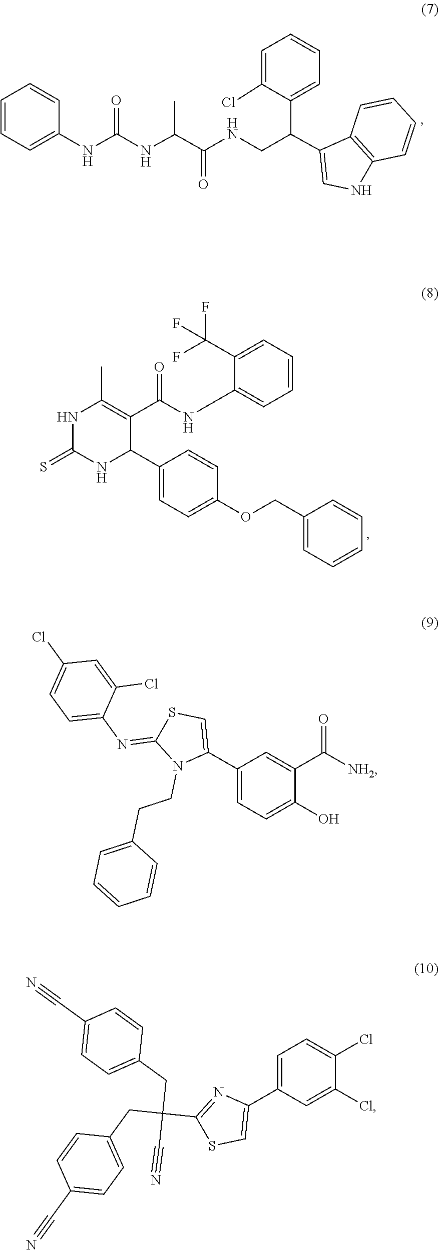 [SMILES: CC(NC(=O)Nc1ccccc1)C(=O)NCC(c1ccccc1Cl)c1c[nH]c2ccccc12 ; CC1=C(C(=O)Nc2ccccc2C(F)(F)F)C(c2ccc(OCc3ccccc3)cc2)NC(=S)N1 ; NC(=O)c1cc(-c2cs/c(=N\c3ccc(Cl)cc3Cl)n2CCc2ccccc2)ccc1O ; N#Cc1ccc(CC(C#N)(Cc2ccc(C#N)cc2)c2nc(-c3ccc(Cl)c(Cl)c3)cs2)cc1] 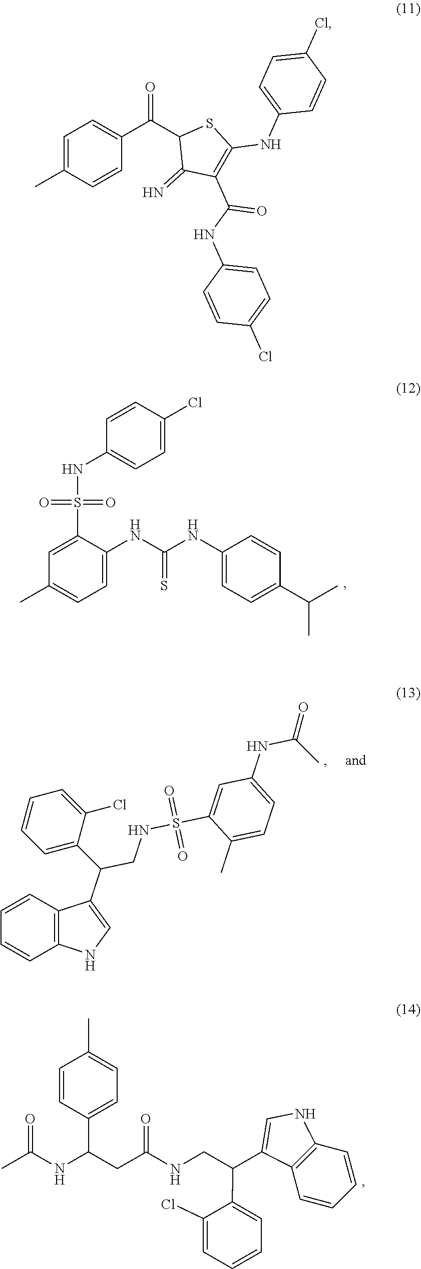
or a compound of formula (I) or (II): 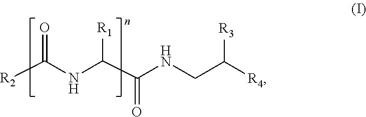
wherein in the compound of formula (I):
n is an integer between 1 and 3;
R1 is a C1-C5alkyl; and
each of R2, R3 and R4, independently of the other is a C6-C10aryl, a C5-C10heteroaryl or a C5-C10carbocycle;
or 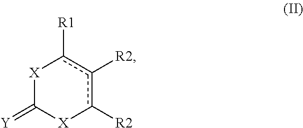
wherein in the compound of formula (II):
each X is a heteroatom selected from O, NH and S;
Y is a heteroatom selected from O, NH and S;
R1 is a C1-C5alkyl;
R2 is a —(C═O)NH—R3;
R3 is a C6-C10aryl or a C5-C10heteroaryl; and
one of the bonds designated is a double bond and the other is a single bond.

15. The composition according to claim 14, for preventing or treating a metabolic syndrome and disorder.

16. The composition according to claim 14, wherein the compound is compound (4) or compound (8).

17. A method of treating a disease or disorder in a subject, the method comprising administering to the subject a compound designated compound (1) through compound (14): 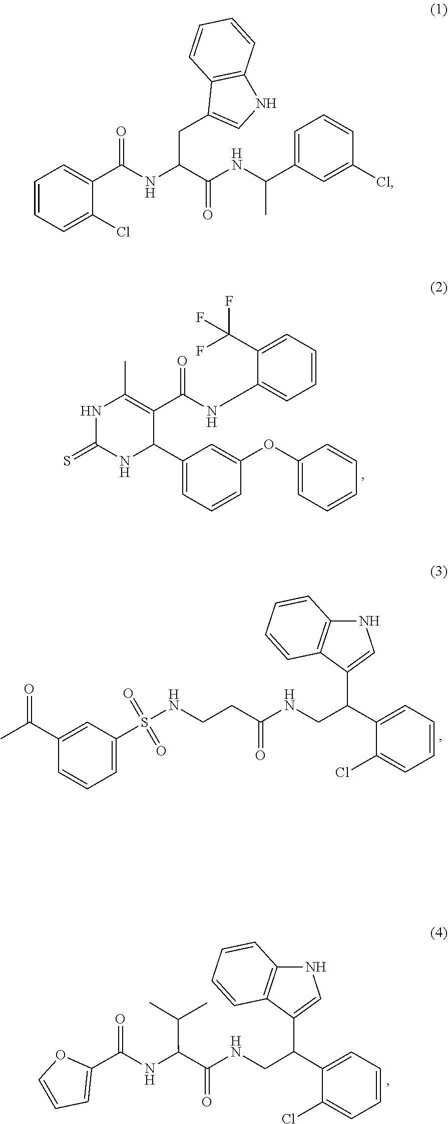 [SMILES: CC(NC(=O)C(Cc1c[nH]c2ccccc12)NC(=O)c1ccccc1Cl)c1cccc(Cl)c1 ; CC1=C(C(=O)Nc2ccccc2C(F)(F)F)C(c2cccc(Oc3ccccc3)c2)NC(=S)N1 ; CC(=O)c1cccc(S(=O)(=O)NCCC(=O)NCC(c2ccccc2Cl)c2c[nH]c3ccccc23)c1 ; CC(C)C(NC(=O)c1ccco1)C(=O)NCC(c1ccccc1Cl)c1c[nH]c2ccccc12] 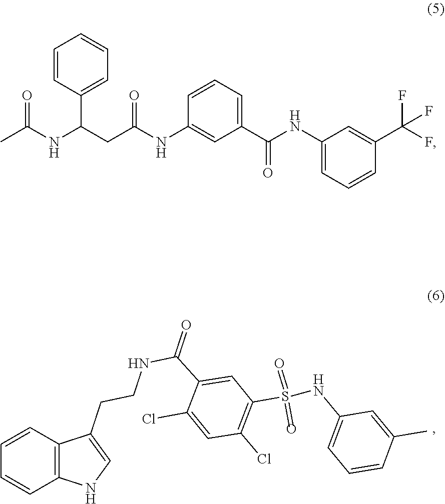 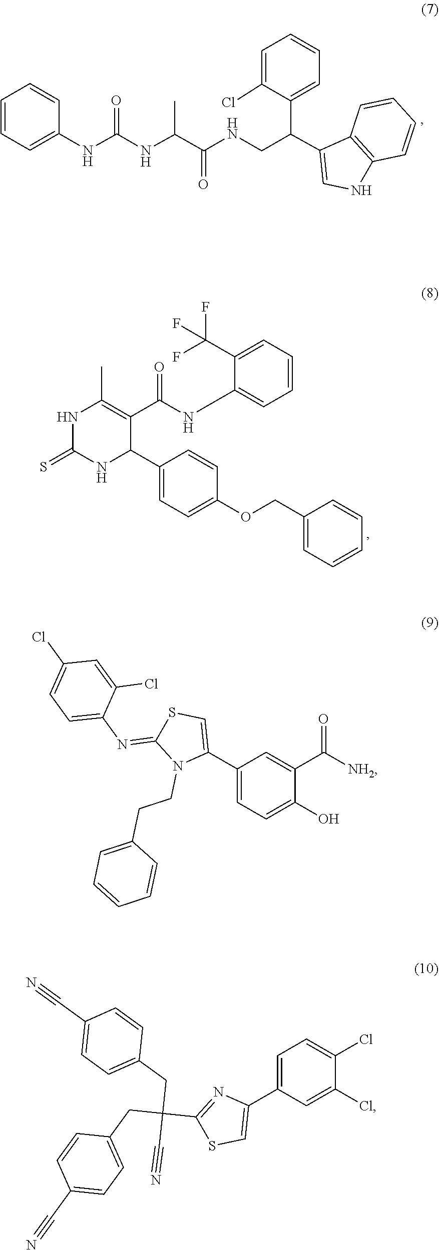 [SMILES: CC(NC(=O)Nc1ccccc1)C(=O)NCC(c1ccccc1Cl)c1c[nH]c2ccccc12 ; CC1=C(C(=O)Nc2ccccc2C(F)(F)F)C(c2ccc(OCc3ccccc3)cc2)NC(=S)N1 ; NC(=O)c1cc(-c2cs/c(=N\c3ccc(Cl)cc3Cl)n2CCc2ccccc2)ccc1O ; N#Cc1ccc(CC(C#N)(Cc2ccc(C#N)cc2)c2nc(-c3ccc(Cl)c(Cl)c3)cs2)cc1] 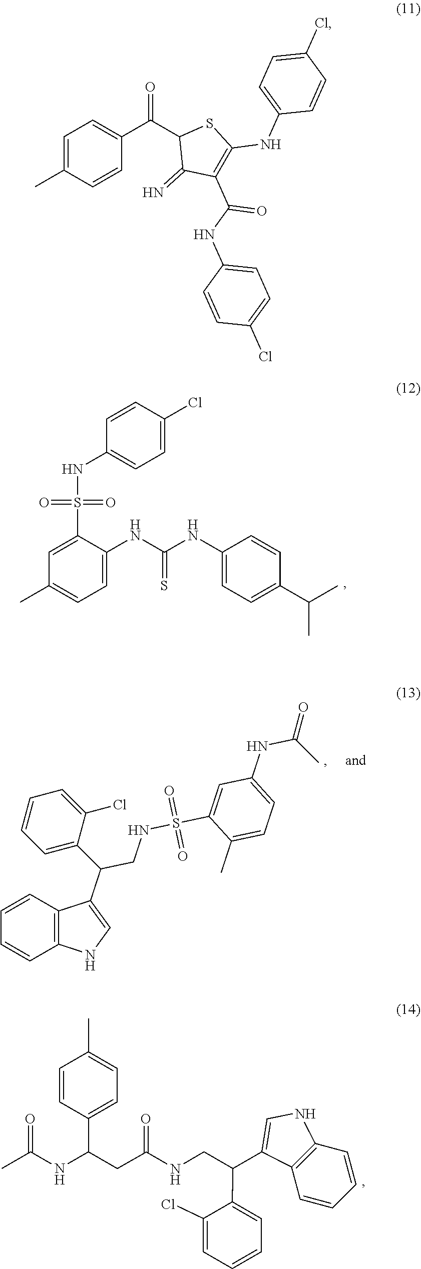
or a compound of formula (I) or (II): 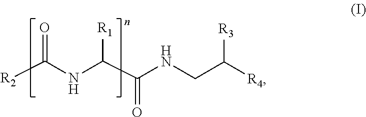
wherein in the compound of formula (I):
n is an integer between 1 and 3;
R1 is a C1-C5alkyl; and
each of R2, R3 and R4, independently of the other is a C6-C10aryl, a C5-C10heteroaryl or a C5-C10carbocycle;
or 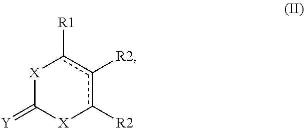
wherein in the compound of formula (II):
each X is a heteroatom selected from O, NH and S;
Y is a heteroatom selected from O, NH and S;
R1 is a C1-C5alkyl;
R2 is a —(C═O)NH—R3;
R3 is a C6-C10aryl or a C5-C10heteroaryl; and
one of the bonds designated is a double bond and the other is a single bond.

18. The method according to claim 17, wherein the compound is compound (4) or compound (8).

19. A compound being a compound designated compound (1) through compound (14): 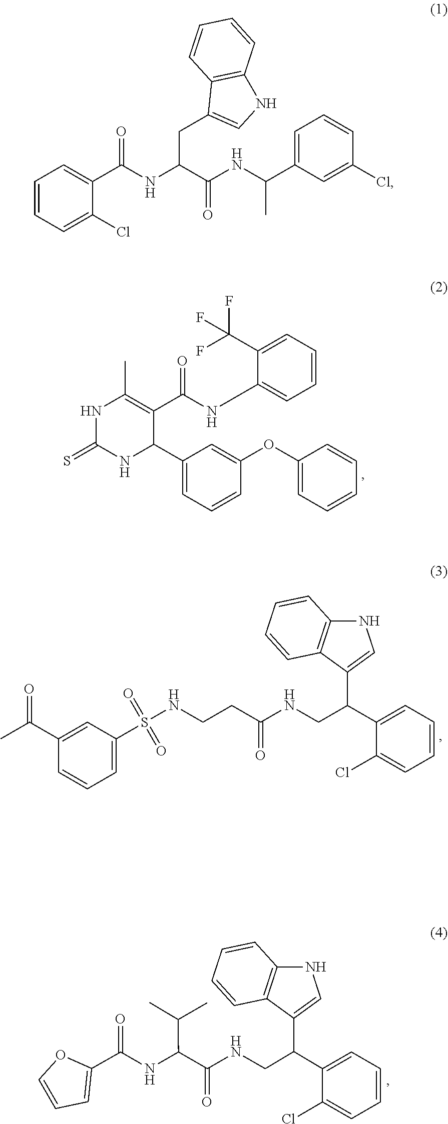 [SMILES: CC(NC(=O)C(Cc1c[nH]c2ccccc12)NC(=O)c1ccccc1Cl)c1cccc(Cl)c1 ; CC1=C(C(=O)Nc2ccccc2C(F)(F)F)C(c2cccc(Oc3ccccc3)c2)NC(=S)N1 ; CC(=O)c1cccc(S(=O)(=O)NCCC(=O)NCC(c2ccccc2Cl)c2c[nH]c3ccccc23)c1 ; CC(C)C(NC(=O)c1ccco1)C(=O)NCC(c1ccccc1Cl)c1c[nH]c2ccccc12] 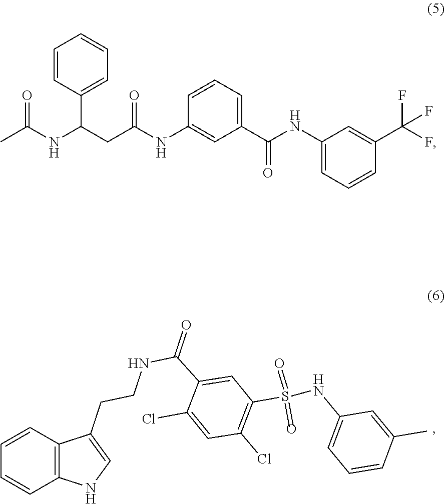 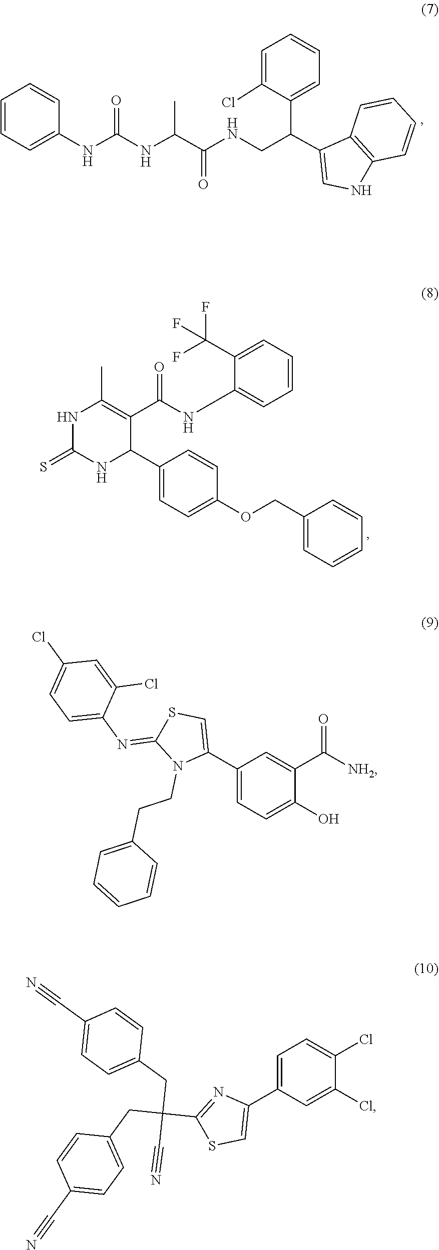 [SMILES: CC(NC(=O)Nc1ccccc1)C(=O)NCC(c1ccccc1Cl)c1c[nH]c2ccccc12 ; CC1=C(C(=O)Nc2ccccc2C(F)(F)F)C(c2ccc(OCc3ccccc3)cc2)NC(=S)N1 ; NC(=O)c1cc(-c2cs/c(=N\c3ccc(Cl)cc3Cl)n2CCc2ccccc2)ccc1O ; N#Cc1ccc(CC(C#N)(Cc2ccc(C#N)cc2)c2nc(-c3ccc(Cl)c(Cl)c3)cs2)cc1] 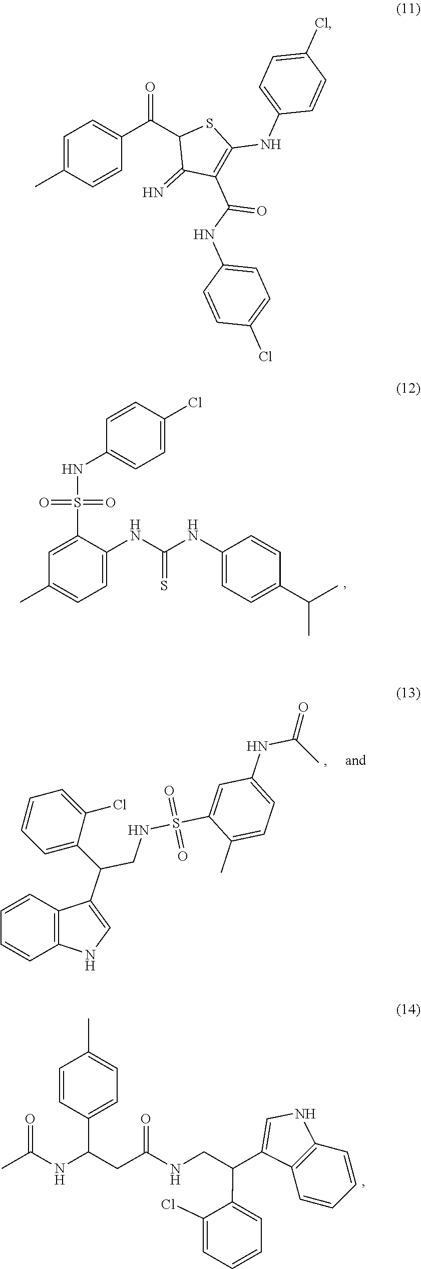
or a compound of formula (I) or (II): 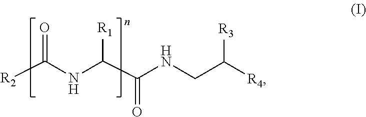
wherein in the compound of formula (I):
n is an integer between 1 and 3;
R1 is a C1-C5alkyl; and
each of R2, R3 and R4, independently of the other is a C6-C10aryl, a C5-C10heteroaryl or a C5-C10carbocycle;
or 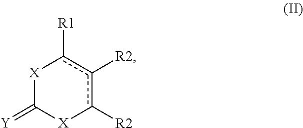
wherein in the compound of formula (II):
each X is a heteroatom selected from O, NH and S;
Y is a heteroatom selected from O, NH and S;
R1 is a C1-C5alkyl;
R2 is a —(C═O)NH—R3;
R3 is a C6-C10aryl or a C5-C10heteroaryl; and
one of the bonds designated [symbol] is a double bond and the other is a single bond;
being:
a modulator of a peripherally restricted CB1/CB2 receptor;
an inhibitor of a peripherally restricted CB1/CB2 receptor;
a neural antagonist of a peripherally restricted CB1/CB2 receptor;
an activator of a peripherally restricted CB1/CB2 receptor;
an inverse agonist of a peripherally restricted CB1/CB2 receptor; or
a blocker of a peripherally restricted CB1/CB2 receptor.

20-30. (canceled)

31. The compound according to claim 19, being compound (4).

32. The compound according to claim 19, being compound (8).
